(19) United States
(12) Patent Application Publication
(10) Pub. No.: US 2014/0302512 A1
(43) Pub. Date: Oct. 9, 2014

(54) GENOTOXICITY TESTING

(71) Applicant: Gentronix Limited, Manchester (GB)

(72) Inventors: Adam Rabinowitz, London (GB); Richard Walmsley, Marple (GB); Matthew Tate, Wakefield (GB)

(73) Assignee: GENTRONIX LIMITED, Manchester (GB)

(21) Appl. No.: 14/053,070

(22) Filed: Oct. 14, 2013

Related U.S. Application Data
(63) Continuation of application No. 13/260,458, filed on Sep. 26, 2011, now abandoned.

(30) Foreign Application Priority Data
Mar. 28, 2009 (GB) 0905410.7

(51) Int. Cl. C12Q 1/68; G01N 33/50

(52) U.S. Cl. CPC C12Q 1/6897; G01N 33/5014
USPC 435/6.13; 435/320.1; 435/366

(57) ABSTRACT

The present invention relates to methods for detecting for the presence of an agent that putatively causes or potentiates DNA damage comprising subjecting a cell (containing a DNA sequence encoding Gaussia luciferase (GLuc) reporter protein operatively linked to a human GADD45α gene promoter and a human GADD45α gene regulatory element arranged to activate expression of the DNA sequence in response to DNA damage) to an agent; and monitoring the expression of the GLuc reporter protein from the cell. The invention also concerns expression cassettes, vectors and cells which may be used according to such a method and also modified media that may be employed in assays and in preferred embodiments of the method of the invention.

### CROSS-REFERENCE TO RELATED APPLICATIONS

The present application is a continuation of U.S. patent application Ser. No. 13/260,458, filed on Sep. 26, 2011, which application is a national phase entry under 35 U.S.C. §371 of International Application No. PCT/GB2010/000581, filed Mar. 26, 2010, published in English, which claims priority from Great Britain Patent Application No. 0905410.7, filed Mar. 28, 2009, all of which are incorporated herein by reference.

### BACKGROUND OF THE INVENTION

The present invention relates to methods for detecting agents that cause or potentiate genome damage, and to molecules and transfected cell lines that may be employed in such methods. In particular, the invention relates to biosensors for detecting genome damage in human cell cultures and other mammalian cell lines.

Genome damage can occur through DNA damage, which is induced by a variety of agents such as ultraviolet light, X-rays, free radicals, methylating agents and other mutagenic compounds. The number of chromosomes in the genome can also be altered, by compounds known as aneugens. DNA damage and/or aneugenesis can also be caused indirectly either by agents that affect enzymes and proteins which interact with DNA (including polymerases and topoisomerases) or by promutagens (agents that can be metabolised to become mutagenic). Any of these agents may cause damage to the DNA that comprises the genetic code of an organism and cause mutations in genes. In animals, such mutations or alterations in chromosome numbers can lead to carcinogenesis or may damage the gametes to give rise to congenital defects in offspring. Such DNA damaging agents can be collectively known as genotoxins.

These DNA damaging agents may chemically modify the nucleotides that comprise DNA, break the phosphodiester bonds that link the nucleotides, or disrupt association between bases (T-A or C-G). Other genome damaging agents may have effects on structural components of DNA (e.g. histones), the mechanisms of nuclear and cell division (e.g. spindle formation), or genome maintenance systems such as topoisomerases and polymerases. To counter the effect of these DNA damaging agents cells have evolved a number of mechanisms. For example, the SOS response in E. coli is a well-characterised cellular response induced by DNA damage in which a series of proteins are expressed, including DNA repair enzymes, which repair the damaged DNA. In mammalians, systems such as nucleotide excision repair and base excision repair mechanisms play a prominent role in DNA damage repair, and are the primary mechanism for removal of bulky DNA adducts and modified bases, whilst non-homologous end-joining and homologous recombination are important in the repair of strand breakage. The majority of these systems also result in cell cycle arrest to allow cells to repair before progressing through cell division.

There are numerous circumstances when it is important to identify what agents may cause or potentiate genome damage. It is particularly important to detect agents that cause genome damage when assessing whether it is safe to expose a person to these agents. For instance, a method of detecting these agents may be used as a genotoxicity assay for screening compounds that are candidate medicaments, food additives or cosmetics to assess whether or not the compound of interest induces genome damage. Alternatively, methods of detecting genome damaging agents may be used to monitor for contamination of water supplies with pollutants that contain mutagenic compounds.

Various methods, such as the Ames Test, the in vitro micronucleus test and the mouse lymphoma assay (MLA), for determining the genotoxicity of an agent are known but are unsatisfactory for a number of reasons. For instance, incubation of samples can take many weeks, when it is often desirable to obtain genotoxic data in a shorter time frame. Furthermore, many known methods of detecting DNA damage (including the Ames Test and related methods) assay lasting DNA damage, as an endpoint, either in the form of mis-repaired DNA (mutations and recombinations) or unrepaired damage in the form of fragmented DNA. However, most DNA damage is repaired before such an endpoint can be measured and lasting DNA damage only occurs if the conditions are so severe that the repair mechanisms have been saturated. DNA damage might be correctly repaired, or inaccurately repaired such that a mutation is created. This mutation endpoint can be measured after DNA repair. Lasting DNA damage such as a DNA double strand break is lethal.

An improved genotoxicity test is disclosed in WO 98/44149, which concerns recombinant DNA molecules comprising a Saccharomyces cerevisiae regulatory element that activates gene expression in response to DNA damage operatively linked to a DNA sequence that encodes a light emitting reporter protein, such as Green Fluorescent Protein (GFP). Such DNA molecules may be used to transform a yeast cell for use in a genotoxicity test for detecting for the presence of an agent that causes or potentiates DNA damage. The cells may be subjected to an agent and the expression of the light emitting reporter protein (GFP) from the cell indicates that the agent causes DNA damage. The genotoxicity tests described in WO 98/44149 detect the induction of repair activity that can prevent an endpoint being reached. The method described in WO 98/44149 may therefore be used to detect for the presence of DNA damaging agents.

U.S. Pat. No. 6,344,324 discloses a recombinant DNA molecule comprising the regulatory element of the hamster GADD153 upstream promoter region that activates gene expression in response to a wide range of cellular stress conditions, linked to a DNA sequence that encodes GFP. This reporter system is carried out in a human head and neck squamous-cell carcinoma cell line. However, problems associated with this reporter system are that it requires at least a four day treatment period at test agent concentrations that result in less than 10% cell survival, followed by analysis of fluorescence by flow cytometry. In addition, the biological relevance of any gene induction when tested with agents at this level of toxicity is debatable. Furthermore, this development does not disclose a means of specifically monitoring for the presence of agents that may cause or potentiate DNA damage, and the mechanism of GADD153 induction remains unclear. Hence, this system is of very limited use as a human DNA damage biosensor.

PCT/GB2005/001913 discloses a recombinant DNA molecule comprising the regulatory element of the human GADD45α gene linked to a light-emitting protein. This reporter system allows rapid high throughput detection of genotoxins within the normal range of toxicity for genotoxicity assays.

### BRIEF SUMMARY OF THE INVENTION

It is an aim of embodiments of the present invention to address problems associated with the prior art, and to provide an improved biosensor for detecting genome damage in human cell cultures.

According to a first aspect of the present invention, there is provided an expression cassette comprising a DNA sequence encoding Gaussia luciferase (GLuc) reporter protein and derivatives thereof, which DNA sequence is operatively linked to a human GADD45α gene promoter and a human GADD45α gene regulatory element arranged to activate expression of the DNA sequence encoding Gaussia luciferase (GLuc) reporter protein in response to genome damage.

By the term “regulatory element”, we mean a DNA sequence that regulates the transcription of a gene with which it is associated, i.e. the DNA sequence encoding the Gaussia luciferase (GLuc) reporter protein.

By the term “operatively linked”, we mean that the regulatory element is able to induce the expression of the GLuc reporter protein.

According to a second aspect of the invention, there is provided a recombinant vector comprising an expression cassette according to the first aspect.

According to a third aspect of the invention, there is provided a cell containing a recombinant vector in accordance with the second aspect of the present invention.

According to a fourth aspect of the present invention, there is provided a method of detecting for the presence of an agent that causes or potentiates genome damage comprising subjecting a cell in accordance with the third aspect of the present invention to an agent; and monitoring the expression of the GLuc reporter protein from the cell.

The method of the fourth aspect of the invention represents a novel cost-effective genotoxicity screen that may be used to provide a pre-regulatory screening assay for use by the pharmaceutical industry and in other applications where significant numbers of agents or compounds need to be tested. It provides a higher throughput and a lower compound consumption than existing in vitro and in vivo mammalian genotoxicity assays, and is sensitive to a broad spectrum of genotoxins.

The method of the fourth aspect of the invention is suitable for assessing whether or not an agent may cause genome damage. By “genome damage” we include agents that affect structural components of DNA (e.g. histones) including histone deacetylation inhibitors, the mechanisms of nuclear and cell division (e.g. spindle formation), or genome maintenance systems such as topoisomerases and polymerases and DNA repair systems. We also include DNA damage, such as the chemical modification of nucleotides or the insertion/deletion/replacement of nucleotides; and alterations in chromosome numbers, and DNA synthesis. Preferably by “genome damage” we mean DNA damage.

It is particularly useful for detecting agents that cause genome damage when assessing whether it is safe to expose a person to genome damaging agents. For instance, the method may be used as a genotoxicity assay for screening whether or not known agents, such as candidate medicaments, pharmaceutical and industrial chemicals, pesticides, fungicides, foodstuffs or cosmetics, induce genome damage. Alternatively, the method of the invention may be used to monitor for contamination of water supplies, leachates and effluents with pollutants containing genome damaging agents.

An existing genotoxicity assay, described in PCT/GB2005/001913, uses a recombinant DNA molecule comprising the regulatory element of the human GADD45α gene linked to GFP, a light-emitting protein. That system allows rapid high throughput detection of genotoxins within the normal range of toxicity for genotoxicity assays using fluorescence spectroscopy.

The inventors decided to develop an alternative genotoxicity assay in which the reporter protein could be detected by bioluminescence. The use of bioluminescence rather than fluorescence to assay reporter protein expression has a number of advantages. Firstly, test compounds that are themselves fluorescent can affect the detection of expression of a fluorescence reporter protein. This would not be a problem if a bioluminescent reporter protein was used, as test compounds are very rarely, if at all, luminescent. Hence the use of a bioluminescent reporter protein will limit any interference caused by fluorescent compounds and reagents in the assay, which means that a greater range of test compounds can be assayed. Also, since the test compounds used in the assay are very rarely luminescent, this means that less control reactions need to be included in a genotoxicity assay using luminescent reporter proteins. Hence a greater number of test compounds can be assayed in parallel. Also, it is not necessary to include a control reaction using a disrupted or mutated luminescent reporter protein.

### BRIEF DESCRIPTION OF THE DRAWINGS

Embodiments of the invention will now be further described, by way of example only, with reference to the following Examples and Figures in which:—

FIG. 1 shows a restriction map of vector (A) pEP-GD532; (B) pHG45-HC plasmid; and (C) pCMV-GLuc-1.

FIG. 2 (A) shows a plasmid map of vector pEP-GD532-GLuc and (B) a diagram of expression cassette GD532-GLuc.

FIG. 3 shows methylnitrosourea (MNU) induction of FLuc, GLuc and GFP reporter protein activity

FIG. 4 shows example data for 4 test compounds on cells having a GADD45α-FLuc expression cassette from an endpoint timecourse experiment.

FIG. 5 shows example data for two test compounds on having a GD532-GLuc expression cassette; (A) a non-genotoxin; (B) a genotoxin.

FIG. 6 shows data from an assay using a highly fluorescent test compound using the GFP reporter protein; (A) GFP data with acridine orange; (B) GLuc data with acridine orange.

FIG. 7 shows results from an assay of a pro-genotoxin with Gluc reporter protein in the presence of S9 extracts; (A) calibration of thiazole orange (TO) with cell number; (B) data from an S9 assay with 6-aminochrysene when the TO cell number is integrated into the assay. The positive decision threshold for +S9 extracts is 1.5, while the positive decision threshold for −S9 extracts is 1.8; both are shown on the graph.

FIG. 8 shows data from a GLuc-based genotoxicity assay using 384-well microtitre plates for the genotoxin 4-nitroquinoline-1-oxide (NQO); (A) relative toxicity curve for NQO measured using the fluorescent cell stain (TO) method described within Example 4; (B) relative GLuc luminescence induction for NQO.

### DETAILED DESCRIPTION OF THE INVENTION

Luciferases are series of enzymes that catalyse light producing chemical reactions in living organisms. They are an example of a bioluminescent reporter protein. Their expression can be monitored using a suitable microplate reader capable of luminescence readings. They can be used in bioluminescence based assays.

Bioluminescence is a form of chemiluminescence that has evolved in various organisms. There are many distinct classes of bioluminescence derived through separate evolutionary histories. These classes are widely divergent in their chemical properties, yet they all undergo similar chemical reactions, namely the formation and destruction of a dioxetane structure. The classes are all based on the interaction of the enzyme luciferase with a luminescent substrate luciferin.

Luciferase genes have been cloned from a very wide range of difference organisms, including, bacteria, beetles (e.g., firefly and click beetle), Renilla, Aequorea, Vargula and Gonyaulax (a dinoflagellate), and crustaceans. There are currently very may different luciferase enzymes that are available for use in bioluminescent assays.

The inventors decided to compare the properties of two different luciferases to GFP in a genotoxicity assay. They wished to determine which luciferase would be the most suitable for use as a bioluminescent reporter protein in a genotoxicity assay. They chose to work with Firefly luciferase (FLuc), which is by far the most commonly used bioluminescent reporter protein. They also chose to study the properties of Gaussia luciferase (GLuc), which was isolated from Gaussia, a calanoid copepod and is not commonly used as a reporter protein in bioluminescent assays.

To their surprise, the inventors identified a number of beneficial characteristics of Gaussia luciferase (GLuc) when used in the genotoxicity assay. When linked to GADD45α gene elements, GLuc accumulates as a signal of genome repair activity in the cell and even persists after the cells have died. Also GLuc persists as a measurable reporter protein when genome repair is complete. In contrast FLuc does not persist as a reporter signal for as long as GLuc. These differences mean that a genotoxicity assay using GLuc can be performed with a single sampling time point to get a measure of the genotoxicity of the test compound, which is not possible with FLuc. The advantages are mainly due to the fact that FLuc is an unstable protein with a short half-life. These advantages of using GLuc rather than FLuc as a reporter protein in a genotoxicity assay were not known and could not have been predicted before the work conducted by the inventors. Indeed, until the present invention GLuc had not been used as a reporter protein for genotoxicity assays.

Furthermore, GLuc protein is secreted from cells, but FLuc protein is not. Hence when FLuc is used as a reporter protein in a genotoxicity assay, cells with FLuc have to be lysed to accurately measure FLuc expression levels. In contrast, GLuc protein is secreted from cells, which means that, when used as a reporter protein in the genotoxicity assay methods below, cells with GLuc do not usually have to be lysed in order to assay GLuc expression levels. Therefore the use of GLuc rather then FLuc as a reporter protein means that cells do not have to be lysed, saving a reagent addition step and incubation step from the assay method.

On the basis of these findings, the inventors have developed a genotoxicity assay in which Gaussia luciferase (GLuc) expression is regulated by GADD45α gene elements. The assay has improvements over existing genotoxicity assays and bioluminescent assays based on FLuc: the assay can be used to measure the genotoxicity of fluorescent test compounds; there is little interference caused by fluorescent compounds and reagents in the assay; the use of GLuc means that the assay can be performed with a single sampling time point to get a measure of the genotoxicity of the test compound.

Additionally, when used in the genotoxicity assay of the method of the invention, GLuc-mediated bioluminescence has an unexpectedly high ‘signal to noise’ ratio, as demonstrated in the accompanying examples. This improved ratio has allowed the inventors to develop a bioluminescence-based genotoxicity assay that uses a lower volume of assay liquid than can be readily used for fluorescence-based assays. As a direct consequence, genotoxicity assays using GLuc-mediated bioluminescence can be performed using 384-well microtitre plates. In contrast, it is difficult to use 384-well microtitre plates for similar fluorescence-based reporter assays, as the reduced volume of assay liquid means a reduced number of cells, and hence a poor ‘signal to noise’ ratio.

Therefore the bioluminescence-based genotoxicity assay of the method of the invention can be more readily used in higher throughput screening systems than with fluorescence-based assays. This may enable the assay to be performed with smaller amounts of test compound and may allow more compounds to be tested per assay microplate.

By the term “Gaussia luciferase (GLuc) reporter protein and derivatives thereof′ we include a protein derived from the marine copepod Gaussia princeps which when expressed is detectable by a luciferase assay. Nucleic acid sequences encoding GLuc proteins are commercially available from a number of different companies; for example, Nanolight (ww.nanolight.com). They are presently not widely used as reporter proteins in assay methods.

Preferably, the Gaussia luciferase (GLuc) reporter protein catalyses the oxidation of coelenterazine in a luminescent reaction.

coelenterazine+O₂→coelenteramide+light

causing the emission of substantial and measurable luminescence.

Nucleotide sequence encoding such a protein can be obtained from a number of difference sources; for example GenBank accession number AY015993.

Derivatives of GLuc include DNA sequences encoding for polypeptide analogues or polypeptide fragments of GLuc, which retain luminescent activity.

Nucleic acid encoding a “humanised” Gaussia luciferase (GLuc) reporter protein maybe obtained from the plasmid obtainable from Nanolight (www.nanolight.com). The nucleic acid sequence of the “humanised” GLuc gene has been optimised for expression in human cell lines. An example of a DNA sequence encoding Gaussia luciferase (GLuc) is shown at positions 2641-3198 of SEQ ID NO:1 at the end of the examples section of the specification. Hence a preferred embodiment of the invention is wherein the Gaussia luciferase (GLuc) reporter protein is encoded by the nucleotide sequence shown at positions 2641-3198 of SEQ ID NO:1.

GLuc produces a high quantum yield of light, does not require ATP and is readily detectable by commercially available luminometers. Cells according to the third aspect of the invention, which contain DNA molecules coding GLuc reporter proteins, may be used according to the method of the fourth aspect of the invention.

Surprisingly, the use of a human GADD45α gene regulatory element in addition to the human GADD45α gene promoter in the expression cassette according to the first aspect of the invention radically enhances the response of the cassette to genotoxic stress and, hence, genome damage in the cell according to the third aspect. Advantageously, the cassette can be analysed for expression of the reporter protein within or after only 48 hours simply by assaying for the activity of the reporter protein in a test culture. The cells may be subjected to the test agent or compound, and expression of the reporter protein in the cell indicates whether the test agent causes genome damage.

The inventors have found that DNA encoding a human GADD45α gene promoter and a human GADD45α gene regulatory element may be operatively linked to a reporter protein to form a cassette according to the first aspect of the invention and then advantageously used in a genotoxic test according to the fourth aspect of the invention. Such cassettes may comprise the whole of the GADD45α gene (including coding sequences) provided that it is operatively linked to DNA encoding a GLuc reporter protein. For instance cassettes may be made according to the first aspect of the invention comprising the whole of, or substantially all of, the GADD45α gene (comprising regulatory elements and promoter) with DNA encoding a GLuc reporter inserted 3′ of the GADD45α promoter (e.g. within the GADD45α coding sequence or at the 3′ of the coding sequence) and arranged to activate expression of the DNA sequence encoding the GLuc reporter protein in response to genome damage.

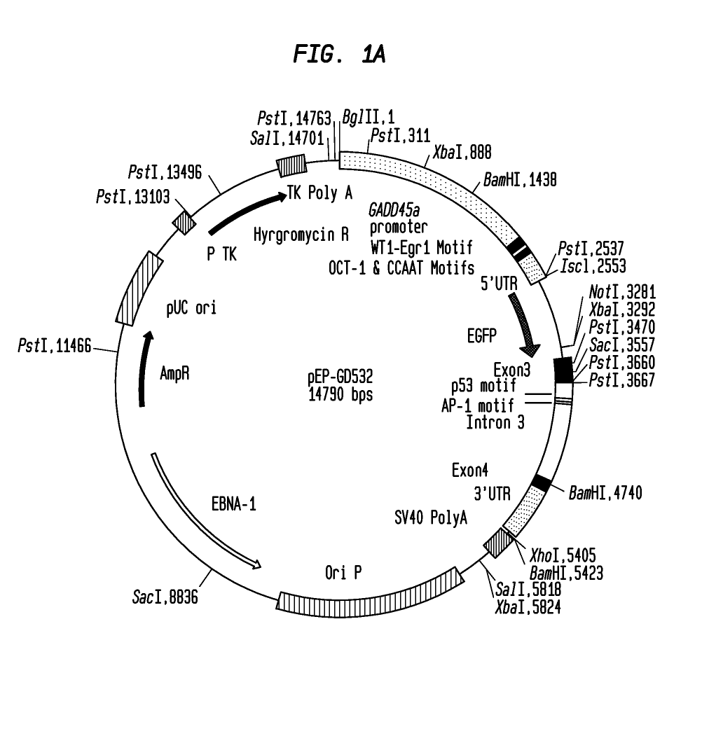

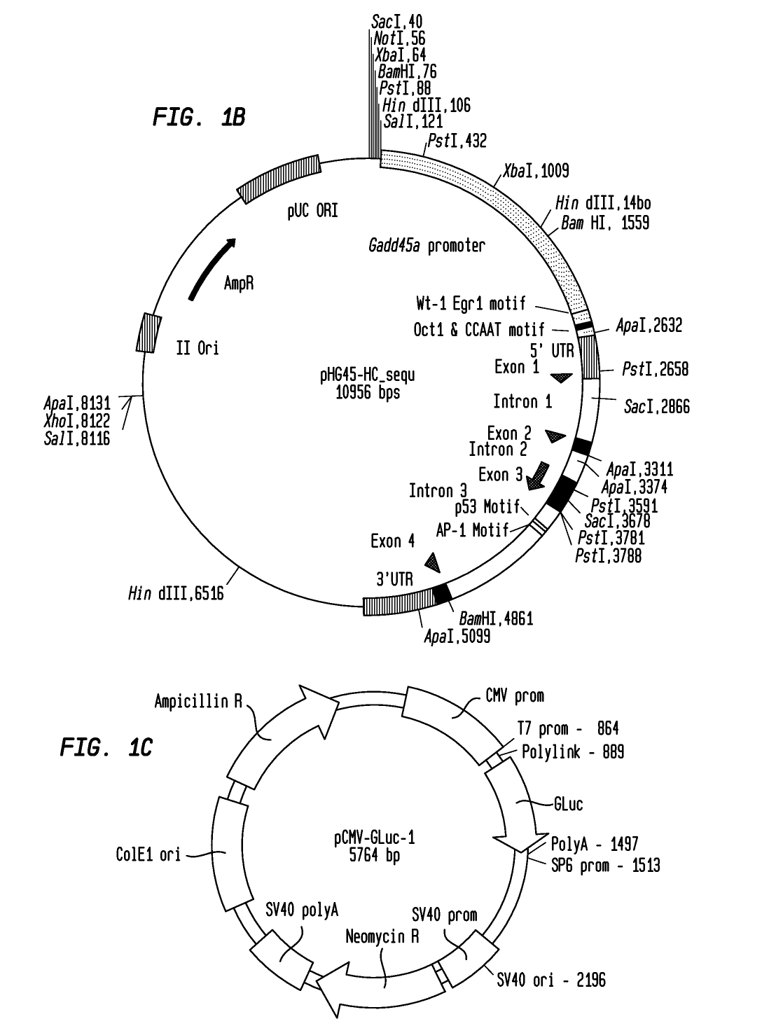

Preferably, the human GADD45α gene promoter sequence induces RNA polymerase to bind to the DNA molecule and start transcribing the DNA encoding the GLuc reporter protein. It is preferred that the promoter sequence comprises the human GADD45α gene promoter sequence and the 5′ untranslated region. The promoter sequence may be obtained from the pHG45-HC plasmid, which is illustrated in FIG. 1. The nucleotide sequence of the GADD45α gene promoter is shown as nucleotides 97 to 2640 of SEQ ID NO: 1 at the end of the examples. It will be appreciated that the promoter may comprise each of the bases 97-2640 or alternatively may be a functional derivative or functional fragment thereof. Functional derivatives and functional fragments may be readily identified be assessing whether or not transcriptase will bind to a putative promoter region and will then lead to the transcription of the marker protein. Alternatively such functional derivatives and fragments may be examined by conducting mutagenesis on the GADD45α promoter, when in natural association with the GADD45α gene, and assessing whether or not GADD45α expression may occur.

The regulatory element in the expression cassette according to the invention may comprise sequences downstream of the GADD45α gene promoter sequence. The regulatory element may comprise functional DNA sequences such as those encoding translation initiation sequences for ribosome binding or DNA sequences that bind transcription factors which promote gene expression following genome damage.

Preferably the term “regulatory element” does not include the GADD45α gene promoter sequence. By “regulatory element” wre include intragenic sequence of the GADD45α gene.

The regulatory element in the expression cassette according to the invention may comprise at least one exon of the GADD45α gene. For example, the regulatory element may comprise Exon 1, Exon 2, Exon 3, and/or Exon 4 of the GADD45α gene, or at least a region thereof, or any combination thereof. Hence, the regulatory element may comprise any combination of the four exons of the GADD45α gene, or at least a region thereof.

In a preferred embodiment, the regulatory element comprises at least a region of Exon 1 of the GADD45α gene, and preferably at least a region of Exon 3 of the GADD45α gene, and more preferably, at least a region of Exon 4 of the GADD45α gene. It is especially preferred that the regulatory element comprises all of Exon 1 of the GADD45α gene, and preferably at least a region of Exon 3 of the GADD45α gene, and more preferably, all of Exon 4 of the GADD45α gene.

The nucleotide sequence of Exon 3 of the GADD45α gene is shown as bases 3325-3562 in SEQ ID No 1. The nucleotide sequence of Exon 4 of the GADD45α gene is shown as bases 4636-5311 in SEQ ID No. 1 in the sequence listing.

Alternatively, or additionally, the regulatory element may comprise a non-coding DNA sequence, for example, at least one intron of the GADD45α gene. For example, the regulatory element may comprise Intron 1, Intron 2, and/or Intron 3 of the GADD45α gene, or at least a region thereof, or any combination thereof. Hence, the regulatory element may comprise any combination of the three introns of the GADD45α gene, or at least a region thereof.

In a preferred embodiment, the regulatory element in the expression cassette according to the invention comprises at least a region of Intron 3 of the GADD45α gene. The nucleotide sequence of Intron 3 of the GADD45α gene is shown as bases 3563-4635 in SEQ ID No. 1 in the sequence listing.

In a preferred embodiment, the expression cassette in accordance with the invention comprises the promoter sequence of the GADD45α gene and also gene regulatory elements found within Intron 3 of the genomic GADD45α gene sequence itself. While the inventors do not wish to be bound by any hypothesis, they believe that Intron 3 of the GADD45α gene, contains a putative p53 binding motif, and that it is this p53 motif which surprisingly enhances the response of the expression cassette to genotoxic stress. The putative p53 binding motif is shown as nucleotide bases 3746-3765 in SEQ ID No. 1 in the sequence listing.

The inventors also believe that Intron 3 of the GADD45α gene may contain a putative TRE motif, which may encode a AP-1 binding site. The putative TRE motif is shown as nucleotide bases 3795-3801 in SEQ ID No. 1 in the sequence listing. Hence, while the inventors do not wish to be bound by any hypothesis, they postulate that this putative AP-1 binding site may also contribute to the improved response to genotoxic agents.

It is preferred that the expression cassette comprises at least the p53 binding motif and/or the AP-1 binding motif from Intron 3 of the GADD45α gene.

The regulatory element may comprise a 3′ untranslated (UTR) region of the GADD45α gene, the nucleotide sequence of which is shown as bases 4750-5311 in SEQ ID No. 1. While the inventors do not wish to be bound by any hypothesis, they believe that this 3′ UTR may be involved with stabilisation of mRNA cassette, and hence, may be surprisingly important when used with the rest of the regulatory element, such as Intron 3.

Hence, preferred expression cassettes according to the first aspect of the invention comprise a human GADD45α gene regulatory element and human GADD45α gene promoter operatively linked to a DNA sequence encoding a Gaussia luciferase (GLuc) reporter protein. Most preferred expression cassettes comprise a human GADD45α gene promoter operatively linked to a DNA sequence encoding a Gaussia luciferase (GLuc), and Intron 3 of the GADD45α gene.

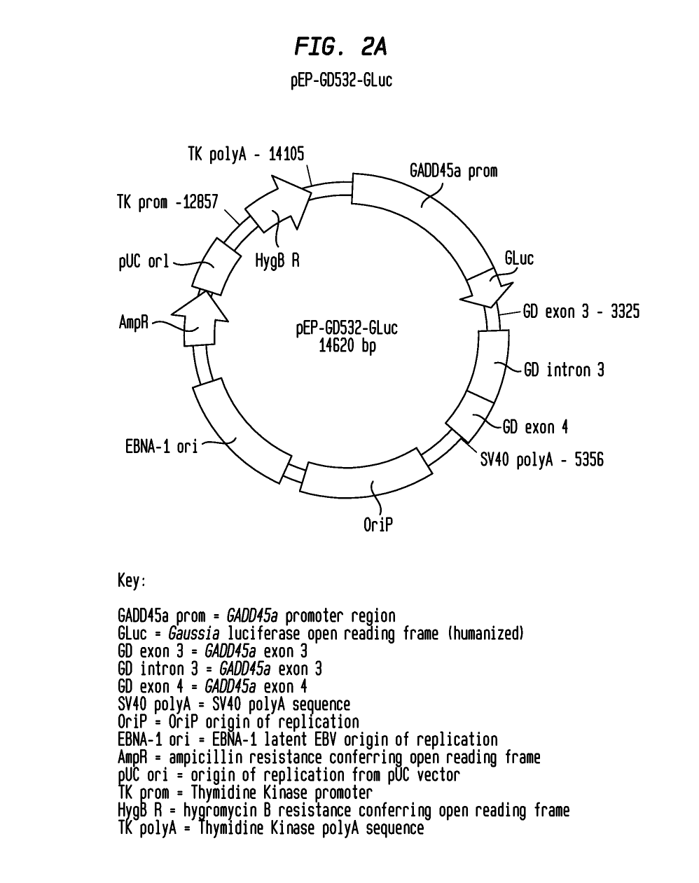

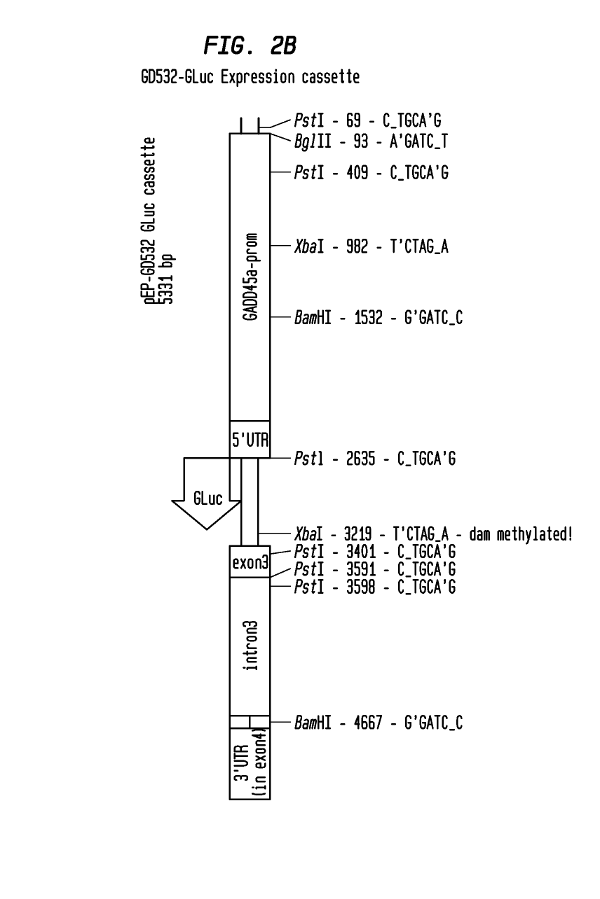

In a further embodiment, the expression cassette according to the first aspect is preferably GD532-GLuc, as shown in FIG. 2. The nucleotide sequence of expression cassette GD532-GLuc is given in SEQ ID No.2 and correspond to nucleotide positions 97 to 5311 of SEQ ID NO:1.

The recombinant vector according to the second aspect of the present invention may for example be a plasmid, cosmid or phage. Such recombinant vectors are of great utility when replicating the expression cassette. Furthermore, recombinant vectors are highly useful for transfecting cells with the expression cassette, and may also promote expression of the reporter protein.

Recombinant vectors may be designed such that the vector will autonomously replicate in the cytosol of the cell or can be used to integrate into the genome. In this case, elements that induce DNA replication may be required in the recombinant vector. Suitable elements are well known in the art, and for example, may be derived from pCEP4 (Invitrogen, 3 Fountain Drive, Inchinnan Business Park, Paisley, PA4 9RF, UK) pEGFP-N1 (BD Biosciences Clontech UK, 21 In Between Towns Road, Cowley, Oxford, OX4 LY, United Kingdom) or pCI and pSI (Promega UK ltd, Delta house, chilworth Science Park, Southampton SO16 7NS, UK).

Such replicating vectors can give rise to multiple copies of the DNA molecule in a transformant and are therefore useful when over-expression (and thereby increased light emission) of the GLuc reporter protein is required. In addition, it is preferable that the vector is able to replicate in human, primate and/or canine cells. It is preferred that the vector comprises an origin of replication, and preferably, at least one selectable marker. The selectable marker may confer resistance to an antibiotic, for example, hygromycin or neomycin. A suitable element is derived from the pCEP4 plasmid (Invitrogen, 3 Fountain Drive, Inchinnan Business Park, Paisley, PA4 9RF, UK).

In a first embodiment, the recombinant vector according to the second aspect is preferably pEP-GD532-GLuc, as illustrated in FIG. 2 and as provided in SEQ ID NO:1.

According to a third aspect of the invention the expression cassette or recombinant vector of the invention is incorporated within a cell. It is preferred that the cell is eukaryotic. Such host cells may be mammalian derived cells and cell lines. Preferred mammalian cells include human, primate, murine or canine cells. The host cells may be lymphoma cells or cell lines, such as mouse lymphoma cells. The host cells may be immortalised, for example, lymphocytes.

Preferred host cells are human cell lines. Preferably, the host cells are human lines having a fully functional p53, for example, ML-1 (a human myeloid leukaemia cell line with wild-type p53; ECACC accession number 88113007), TK6 (a human lymphoblastoid cell line with wild-type p53; ECACC accession number 95111725). However, host cell lines of WI-L2-NS (ECACC accession number 90112121) and WTK1 (both of which are sister lines of TK6 and have mutant p53 proteins) are also envisaged. Hep G2 (ECACC accession number 85011430) and HepaRG (BioPredic; http://pagesperso-orange.fr/biopredic/index.html), both of which are human hepatoma derived cell lines, can also be used. (ECACC General Office, CAMR, Porton Down, Salisbury, Wiltshire, SP4 OJG, United Kingdom).

The inventors have found that TK6 human cells are particularly preferred cell lines for use according to the method of the invention. While the inventors do not wish to be bound by any hypothesis, they believe that TK6 cells are most useful because they have a fully functional p53.

Host cells used for expression of the protein encoded by the DNA molecule are ideally stably transfected, although the use of unstably transfected (transient) cells is not precluded.

Transfected cells according to the third aspect of the invention may be formed by following procedures described in the Example. The cell is ideally a human cell line, for example TK6. Such transfected cells may be used according to the method of the fourth aspect of the invention to assess whether or not agents induce or potentiate DNA damage. GLuc expression is induced in response to DNA damage and the light emitted by GLuc may be easily measured using known appropriate techniques.

Most preferred cells according to the third aspect of the invention are TK6 cells transformed with the vector pEP-GD532-GLuc. These cells are referred to herein as GLuc-T01.

It is also envisaged that the expression cassette according to the invention may be integrated into the genome of a host cell. The skilled technician will appreciate suitable methods for integrating the cassette into the genome. For example, the expression cassette may be harboured on a retroviral vector, which in combination with a packaging cell line may produce helper-free recombinant retrovirus, which may then be introduced into the host cell. The cassette may then integrate itself into the genome. Examples of suitable helper-free retroviral vector systems include the pBabePuro plasmid with the BING retroviral packaging cell line [Kinsella and Nolan, 1996. Episomal Vectors Rapidly and Stably Produce High-Titer Recombinant Retroviruses. Human Gene Therapy. 7:1405-1413.]

The method of the fourth aspect of the invention is particularly useful. for detecting agents that induce genome, particularly DNA damage, at low concentrations. The methods may be used to screen compounds, such as candidate medicaments, food additives or cosmetics, to assess whether it is safe to expose a living organism, particularly people, to such compounds. Alternatively, the method of the fourth aspect of the invention may be employed to detect whether or not water supplies are contaminated by genome damaging agents or agents that potentiate genome damage. For instance, the methods may be used to monitor industrial effluents for the presence of pollutants that may lead to increased genome damage in people or other organisms exposed to the pollution.

The method of the invention is preferably performed by growing cells transfected with a recombinant vector according to the second aspect of the invention (such as pEP-GD532-GLuc), incubating the cells with the agent which putatively causes genome damage for a predetermined time and monitoring the expression of the GLuc reporter protein directly from a sample of the cells.

Suitable methods of luminescence detection and quantitation will be known to the skilled technician, and a method is described in the Examples.

According to a preferred embodiment of the method of the invention, luminescence readings may be recorded from TK6 cells transfected with pEP-GD532-GLuc, for example, from the well of a microplate. An example of a suitable microplate is a 96 well, white, clear-bottom sterile microplates (Matrix Technologies ScreenMates: catalogue no. 4925 are recommended for optimum performance).

Also, as discussed above due to unexpectedly high ‘signal to noise’ ratio the luminescence-based genotoxicity assay method of the invention can be performed using less assay liquid (and hence fewer cells and less test compound) than can be readily used for fluorescence-based assays. As a direct consequence, the method of the invention can be performed using 384-well microtitre plates. Hence a further example of a suitable microplate is a 384 well, black, sterile microplate; suitable plates are also available from Matrix Technologies ScreenMates.

Luminescence and absorbance measurements may be recorded using a suitable microplate reader, for example, Tecan Infinite F500 with injectors

Most preferred protocols for conducting the method of the fourth aspect of the invention are described in the accompanying Examples.

There may be background (“constitutive”) expression of GLuc from the GADD45α-GLuc constructs, thus the higher the cell density, the more luminescent the culture. In order to correct for any luminescent increase that is consequent on growth, the luminescent data are divided by absorbance data (cell density) to give ‘brightness units’, i.e. the measure of average luminescence per cell. This is independent of culture density. Accordingly, measurement of absorbance may be used primarily for normalisation of luminescent signals rather than a measurement of the genotoxicity of the test agent. Accordingly, it is envisaged that a secondary assay may be used in conjunction with the absorbance measurement in order to determine toxicity via cell viability and apoptosis. For example, using the Biovision Bioluminescence Cytotoxicity Assay (Biovision Incorporated, 2455-D Old Middlefield Way, Mountain View, Calif. 94043, USA), or the Vybrant® Apoptosis Assay Kit (Molecular Probes Inc., 29851 Willow Creek Road, Eugene, Oreg. 97402, USA).

Preferred methods according to the fourth aspect of the invention will utilise cells according to the third aspect of the invention (e.g. GLuc-T01).

It will be appreciated that some non-genotoxic compounds can be chemically altered by cellular metabolism. In mammals this process is often called metabolic activation (MA). MA can convert certain non-genotoxic compounds (for example promutagens) into genotoxic compounds. Most frequently MA occurs in the liver. For this reason it is often preferred that genotoxicity tests are adapted such that assays of test compound are carried out in the presence and absence of liver extracts that are capable of metabolising a compound as if it were being metabolised in vivo. Example 4 illustrates a preferred method according to the fourth aspect of the invention which utilises a liver extract (known to the skilled person) called S9. Inclusion of such an extract allows assays to detect compounds that only become genotoxic after passage through the liver.

When S9 liver extract is used in the method of the invention, it is preferred that the density of the cells in the population is determined using a cell stain. This is because the inventors have determined that, as described further in Example 4, relative insensitivity of the optical absorbance measurement used to estimate cell density was found to result in reduced sensitivity of the assay for pro-genotoxins in S9 metabolic activation studies.

As discussed in more detail in Example 2 below, it is useful to have clear definitions of positive and negative results from routine assays and such definitions have been derived, taking into account the maximum noise in the system and data from chemicals where there is a clear consensus on genotoxicity and mechanism of action.

Where the assay includes S9 liver extracts, the genotoxic threshold is set at a relative GLuc induction of 1.5 (i.e. a 50% increase). Hence a positive genotoxicity result (+) is concluded if a test compound produces a relative GLuc induction greater than the 1.5 threshold.

Where the assay does not include S9 liver extracts, the genotoxic threshold is set at a relative GLuc induction of 1.8 (i.e. an 80% increase). Hence a positive genotoxicity result (+) is concluded if a test compound produces a relative GLuc induction greater than the 1.8 threshold.

Also, within the field of genetic toxicology it is occasionally desirable to assess assay results in a way that acknowledges variations in potency of genotoxic effect between different compounds. Hence, GLuc inductions may also be assessed using the following criterion: a positive (+) genotoxicity result is concluded if one or more test compound concentrations yields a luminescence induction greater than the 1.5 or 1.8 threshold. A negative genotoxicity result (−) is concluded where no compound dilutions produce a relative GLuc induction greater than the 1.5 or 1.8 threshold.

The inventors subsequently discovered that a fluorescent cell stain could be used to replace the optical absorbance measure. This is because the two methods are effectively different ways of estimating the same thing. Surprisingly, the method by which they used the cell stain improved the sensitivity of cell number estimation and hence the detection of pro-genotoxins.

Preferably the cell stain used in the adapted protocol is a cyanine dye, more preferably thiazole orange (TO) which is a cyanine dye that binds to DNA and RNA. The binding of TO to DNA greatly enhances its fluorescence intensity, allowing for its detection without the need to wash away background, unbound TO.

Preferably in the method of the fourth aspect of the invention the expression of the GLuc reporter protein is monitored after between 46 to 50 hours from exposure to the test compound; most preferably after 48 hours.

In some embodiments of the fourth aspect of the invention, the method of detecting for the presence of an agent that causes or potentiates genome damage includes a step of monitoring the expression of the GLuc reporter protein from a cell. The GLuc reporter protein catalyses the oxidation of the substrate coelenterazine in a luminescent reaction. The inventors have determined that in some reaction conditions (particular when a number of reactions are serially performed) coelenterazine can be unstable such that a degree of variation can be introduced to the luminescence signal, which can affect the sensitivity and robustness of the assay.

On further investigation, the inventors determined that coelenterazine can be stabilised by the presence of an oxidising agent, such as ascorbic acid (vitamin C). Alternatively, coelenterazine can be stabilised by the presence of tris(hydroxymethyl)aminomethane (TRIS), preferably at pH 7.4 and at a final concentration of 100 mM. Moreover, coelenterazine can be further stabilised by the presence of β-Cyclodextrin.

Hence a preferred method of the invention is wherein the coelenterazine is prepared as a 5 mM stock solution in acidified methanol. A Luminescence Buffer is prepared (400 mM Tris-HCl; 5 mM β-Cyclodextrin; Deionised water; buffered to pH 7.4 with 10 N NaOH). The stock coelenterazine solution is then diluted 2000-fold in the luminescence buffer to give 2.5 μM coelenterazine solution buffered to pH to 7.4 by TRIS). This is the injection solution which is added to the reaction assay (leading to a further 4-fold dilution of coelenterazine).

All of the features described herein (including any accompanying claims, abstract and drawings), and/or all of the steps of any method or process so disclosed, may be combined with any of the above aspects in any combination, except combinations where at least some of such features and/or steps are mutually exclusive.

### Example 1

### Cloning of pEP-GD532-GLuc

### Summary

To exchange the GFP ORF for a Gaussia luciferase (GLuc) ORF in the GADD45α reporter construct.

### Protocol

The Gaussia luciferase ORF was cloned from the plasmid pCMV-GLuc-1 (Nanolight) using PCR. The pCMV-GLuc-1 plasmid is sold commercially by NEB as pCMV-GLuc. A plasmid map of pCMV-GLuc-1 is provided in FIG. 1. The PWO high-fidelity polymerase (Roche) was used to minimise the production of PCR induced mutations. The forward and reverse primers contained 8 additional (non-complementary) nucleotides encoding the recognition sequences for the restriction endonucleases XhoI and Nod respectively. The protocol for the PCR reaction is shown below.

### Primers:

| Name | Sequence 5′-3′ | Tm | SEQ ID No. |
|---|---|---|---|
| GLuc-F | gggtcgagagtcaaag | 50.4° C. | 3 |
| | ttctgtttgccctg | (69.9° C.) | |
| GLuc-R | gcggccgcattagtca | 50.2° C. | 4 |
| | ccaccggcccc | (77.9° C.) | |

### Reaction Mix:

| Reagent | Volume |
|---|---|
| dNTP mix (10 mM of each) | 1 μl |
| pGLuc-F (10 μM) | 3 μl |
| pGLuc-R (10 μM) | 3 μl |
| 10x PWO PCR buffer (+20 mM MgSO₄) | 5 μl |
| pCMV-GLuc (miniprep) | 1 μl |
| PWO polymerase (5 U/μl) | 0.4 μl |
| ddH₂0 | 36.6 μl |

### PCR Reaction Conditions:

| Conditions | Cycle Number |
|---|---|
| 94° C.-2 min | 1x |
| 94° C.-20 s | 10x |
| 45° C.-30 s | |
| 72° C.-60 s | |
| 94° C.-25 s | 8x |
| 65° C.-30 s | |
| 72° C.-60 s + 5 s/cycle | |
| 72° C.-4 minutes | 1x |
| 4° C.-Soak | |

The PCR products were cleaned and the 5′ termini phosphorylated using T4 polynucleotide kinase (NEB). The plasmid pBluescript II SK (−) was linearised using the EcoRI site and the blunt ended PCR product was ligated into the plasmid.

The pEP-GD532 plasmid (FIG. 1) was cut and linearised with AscI and the resultant 5′ overhangs were removed with the Mung bean nuclease enzyme. The GFP ORF was then removed from the linearised plasmid using a NotI digest and the pEP-GD532 plasmid backbone was separated and cleaned using agarose gel electrophoresis and gel extraction. The cloning and sequence of pEP-GD532 plasmid is fully described in PCT/GB2005/001913.

The pBluescript II SK (−) plasmid containing the GLuc PCR product was cut with XhoI and the resultant 5′ overhangs were removed with the Mung bean nuclease enzyme and the resultant DNA product(s) were cleaned. The DNA was then subjugated to digestion with NotI and the released GLuc PCR product was separated and cleaned using agarose gel electrophoresis.

The purified GLuc ORF was then cloned into the pEP-GD532 backbone using the sticky ends generated by the NotI digestion and the blunt ends generated by the XhoI and AscI digestion followed by Mung bean nuclease treatment. This generated the GADD45α reporter vector pEP-GD532-GLuc, as shown in FIG. 2.

Sequence Information for pEP-GD532-GLuc

The nucleic acid sequence (SEQ ID NO:1) of pEP-GD532-GLuc plasmid is provided in Annex 1 at the end of the accompanying examples. Significant nucleic acid sequences within the pEP-GD532-GLuc plasmid are listed below.

Sequence Annotation of pEP-GD532-GLuc Plasmid Shown in Annex 1:

| Motif | Position |
|---|---|
| GADD45α promoter | 97-2640 |
| Gaussia Luciferase open reading frame | 2641-3198 |
| GADD45α exon 3 | 3325-3562 |
| GADD45α intron 3 | 3563-4635 |
| GADD45α exon 4 | 4636-5311 |
| SV40 poly A | 5356-5597 |
| OriP origin of replication | 6018-7993 |
| EBNA-1 latent EBV origin of | 8294-10219 |
| replication ORF | |
| Ampicillin resistance ORF | 10845-11705 |
| pUC origin of replication | 11714-12489 |
| Thymidine kinase promoter | 12857-13019 |
| Hygromycin resistance ORF | 13083-14093 |
| Thymidine kinase poly A | 14105-14376 |

Cell Line Having the pEP-GD532-GLuc Plasmid

TK6 cells are transfected with pEP-GD532-GLuc by electroporation using a method adapted from Xia and Liber [Methods in Molecular biology, Vol. 48: Animal Cell Electroporation and Electrofusion Protocols, 1995. Edited by J. A. Nickoloff. Humana Press Inc., Totowa, N.J., USA, Pages 151-160], and clones bearing the reporter plasmids are selected. The cell line selected for further work is called GLuc-T01.

### Example 2

### Protocol for a Genotoxicity and Cytotoxicity Assay Using GLuc

The inventors have developed a preferred assay for measuring genotoxicity and cytotoxicity of a test compound using cell line GLuc-TO1 which has the pEP-GD532-GLuc plasmid.

The assay has the following steps, as further described below: (1) preparing a microplate for use in an assay; (2) conducting the assay in the microplates; (3) collecting and analysing the data; and (4) making a judgment on genome damage and the consequences.

The assay is performed using a microplate reader capable of luminescence and absorbance readings, equipped with injectors capable of single well additions.

### 2.1 Microplates

Assays are carried out in white, clear-bottom, 96 well, sterile microplates (Matrix Technologies ScreenMates: catalogue no. 4925 is recommended for optimum performance). Black, clear-bottom, 96 well, sterile microplates can also be used (Matrix Technologies ScreenMates: catalogue no. 4929)

### 2.2 Assay

Using the standard dilution protocol described here, all concentrations are halved in the microplate well when a sample volume of 75 μl is combined with 75 μl of cell culture. All standard and test chemicals and reagents should be prepared fresh shortly before the assay is performed.

Diluent—2% (v/v) DMSO in sterile water.

### 2.2.1 Preparation of Test Compound

The final concentration of each test compound must be in a solution that matches the diluent used, typically 2% v/v DMSO in sterile water, such that the diluent solvent itself is not diluted across the plate.

An initial concentration of 2 mM or 1 mg/ml (whichever is lowest) is recommended (equating to 1 mM or 500 μg/ml of test compound on the microplate). It is desirable that the test compound is fully soluble at the top concentration tested. A minimum of 250 μl of each test compound is required per plate. The recommended method to prepare solutions of test compounds is as follows:
For compounds with high aqueous solubility—dissolve directly in aqueous diluent (i.e. 2% DMSO) and dilute, with diluent, as necessary.
For compounds of limited aqueous solubility—dissolve in 100% DMSO, dilute as necessary in 100% DMSO, and then add 20 μl of the DMSO stock standard to 980 μl sterile water to produce a test solution containing 2% v/v DMSO. If the compound precipitates from solution when the DMSO standard is added to water, the original DMSO stock standard can be diluted further with 100% DMSO. The 20 μl+980 μl water dilution step is then repeated to produce a fresh test standard.

### 2.2.2 Preparation of Control Compounds

4-Nitroquinoline 1-oxide (NQO: e.g. Sigma-Aldrich, catalogue no. N8141-250MG) is used as a control compound.

The control compound solutions are prepared in diluent to the following concentrations:
Standard 1—NQO HIGH=1 μg/ml
Standard 2—NQO LOW=0.25 μg/ml

Aliquots of NQO in 100% DMSO can be prepared and frozen down in 20 μl volumes at 50× test concentration, then defrosted immediately prior to use, and 980 μl of water added to achieve the correct test concentration in 2% DMSO.

### 2.2.3 Preparation of the Cells

Standard cell culture methods are used to prepare GLuc-TO 1 cells for use in the assay. The assay requires cells to be in logarithmic growth phase; therefore cultures should have achieved a density of between 5×10⁵ cells/ml and 1.2×10⁶ cells/ml before they can be used in the assay. Cells are grown in routine culture medium:

| | Stock | Final | Volume |
|---|---|---|---|
| Reagent | Concentration | Concentration | (ml) |
| RPMI 1640 + GlutaMAX | — | — | 500 |
| Sodium Pyruvate | 100 mM | 1.8 mM | 10.4 |
| Hygromycin B | 50 mg/ml | 200 μg/ml | 2.3 |
| Pen/Strep | 5,000 IU/ml/ | 50 IU/ml/ | 5.8 |
| | 5,000 μg/ml | 50 μg/ml | |
| Heat Inactivated Donor Horse | 100% | 10% | 57 |
| Serum | | | |

When used, prepare a 10 ml suspension of GLuc-T01 cells at a density of 2×106 cells/ml in Assay Medium (GS-HC-AM).

### Assay Medium:

Working concentrations of components of the Assay Medium are set out below:

| Component | mg/L |
|---|---|
| INORGANIC SALTS: | |
| Ca(N0₃)₂•4H₂0 | 100.00 |
| KCl | 400.00 |
| MgSO₄ (anhyd.) | 48.84 |
| NaCl | 6000.00 |
| NaHCO₃ | 2000.00 |
| Na₂HPO₄ (anhyd.) | 800.00 |
| OTHER COMPONENTS: | |
| D-Glucose | 2000.00 |
| Glutathione (reduced) | 1.00 |
| AMINO ACIDS: | |
| L-Arginine HCl | 241.86 |
| L-Asparagine (free base) | 50.00 |
| L-Aspartic Acid | 20.00 |
| L-Cystine•2HCl | 65.20 |
| L-Glutamic Acid | 20.00 |
| Glycine | 10.00 |
| L-Histidine (free base) | 15.00 |
| L-Hydroxyproline | 20.00 |
| L-Isoleucine | 50.00 |
| L-Leucine | 50.00 |
| L-Lysine•HCl | 40.00 |
| L-Methionine | 15.00 |
| L-Phenylalanine | 15.00 |
| L-Proline | 20.00 |
| L-Serine | 30.00 |
| L-Threonine | 20.00 |
| L-Tryptophan | 5.00 |
| L-Tyrosine (disodium salt) | 28.83 |
| L-Valine | 20.00 |
| VITAMINS: | |
| D-Biotin | 0.20 |
| D-Ca Pantothenate | 0.25 |
| Choline Chloride | 3.00 |
| Folic Acid | 1.00 |
| i-Inositol | 35.00 |
| Nicotinamide | 1.00 |
| Para-aminobenzoic Acid | 1.00 |
| Pyridoxal HCl | 1.00 |
| Thiamine HCl | 1.00 |
| Vitamin B₁₂ | 0.005 |

### 2.2.4 Preparation of the Assay

The following standard protocol may be followed. A stock of a test chemical, or sample containing an agent that putatively caused DNA damage, is prepared in 2% v/v aqueous DMSO as described above, and used to make a dilution series across a 96 well microplate and a ‘control’ (see below). To achieve this, 150 microlitres of the test chemical solution are put into a microplate well. Each sample is serially diluted by transferring 75 microlitres into 75 microlitres of 2% DMSO, mixing, and then taking 75 microlitres out and into the next well. This produces 9 serial dilutions of 75 microlitres each. The final top concentration of test chemical/sample is 1 mM or 500 μg/ml on the microplate.

75 μl of GLuc-T01 cells in Assay Medium (GS-HC-AM) as described above are then added to each well as appropriate.

The following controls are included in the microplate:

a. Blank well.
b. Test compound/sample alone.
c. Assay medium alone.
d. Control compound with cells.
By “blank well” we mean that the control contains the solvent used as the carrier for the test compound, typically 2% DMSO.

Once finished, the microplates are covered with a breathable membrane. The plate is gently shaken for 10 to 15 seconds on a microplate shaker (to fully mix the contents of each well) and then incubated at 37° C., 5% CO₂, 95% humidity, without shaking, for 48 hours. Plates should be incubated and analysed after 48 hours+/−2 hours.

### 2.3 Collecting and Analysing the Data

Plates are first read for absorbance in each well, at a wavelength of ˜620 nm. When reading luminescence, 50 μl of injector solution is added to each well, the plate shaken using the reader facilities and then after an integration time of 3 seconds luminescence is read. An example of a suitable reader and injector system is a Tecan Infinite F500

### 2.3.1 Injector Solution

Acidified Methanol (10 ml)=9.9 ml Methanol and 100 μl of 37% HCl 5 mM Coelenterazine Stock (4.72 ml)=10 mg Coelenterazine (native, MW 423.48, CAS#55779-48-1) dissolved in 4.72 ml of the acidified methanol. Pipette 20 μl aliquots into microfuge tubes and store at −80° C. in the dark.

50 mM β-Cyclodextrin (100 ml)=7.3 g 2-Hydroxypropyl-β-cyclodextrin (0.8 molar substitution, MW 1460, CAS#128446-35-5) and distilled water to 100 ml. Filter sterilise and store at 4° C.

Coelenterazine carrier solution=20 ml of Gentronix Assay Medium, 5 ml 50 mM β-Cyclodextrin and 25 ml of sterile distilled water. If all constituents are sterile then solution may be stored at 4° C. for 2 weeks.

5 mM coelenterazine stock solution in acidified methanol should be added to the carrier solution approximately 30 minutes before the first plate read. A small volume of the carrier solution will be dead volume, used to prime the plate reader injector system as well as used in the actual luminescence read. The following volumes of carrier solution+coelenterazine should be prepared.

| Number of | Coelenterazine | Carrier Solution |
|---|---|---|
| Plates | Stock Vol. | Vol |
| 1 | 6 μl | 12 ml |
| 2-4 | 12 μl | 24 ml |

After preparation, the injector solution should have minimal exposure to light and be kept at room temperature.

As mentioned in above, the assay can also be performed using a coelenterazine solution buffered to pH 7.4. Here the coelenterazine is prepared as a 5 mM stock solution in acidified methanol. A Luminescence Buffer is prepared (400 mM Tris-HCl; 5 mM β-Cyclodextrin; Deionised water; buffered to pH 7.4 with 10 N NaOH). The stock coelenterazine solution is then diluted 2000-fold in the luminescence buffer to give 2.5 μM coelenterazine solution buffered to pH to 7.4 by TRIS). This is the injection solution which is added to the reaction assay (leading to a further 4-fold dilution of coelenterazine).

### 2.3.2 Addition of Injector Solution

The syringe injection speed should be set to high as this ensures that when the coelenterazine solution is injected into the well it is rapid rapidly mixed. The syringe re-fill speed should be set to low, as this ensures that bubbles are not created in the syringe barrel.

### 2.3.3 Data Analysis

Following the 48 hour incubation, luminescence and absorbance data are collected from the microplates. A microplate reader combining luminescence and absorbance functionality is used; by way of example, this reader may be a Tecan Infinite F500 (Tecan UK Ltd.). Luminescence data are collected with an integration time of 3 seconds after injection of the substrate and shaking of the microplate (within the reader). Optical absorbance is measured through a 600 nm or 620 nm filter. These luminescence and absorbance data are transported into a Microsoft Excel spreadsheet, and converted to graphical data. Data processing is minimal: absorbance data give an indication of reduction in proliferative potential and these data are normalised to the vehicle-treated control (=100% growth). Luminescence data are divided by absorbance data to give ‘brightness units’, the measure of average GLuc induction per cell. These data are normalised to the vehicle-treated control (=1). In this way, one can distinguish between a small number of highly luminescent cells and a large number of weakly luminescent cells. The decision (see below), on whether or not a compound is classified as being genotoxic is generated automatically within the software.

It is useful to have clear definitions of positive and negative results from routine assays and such definitions have been derived, taking into account the maximum noise in the system and data from chemicals where there is a clear consensus on genotoxicity and mechanism of action. Naturally it is also possible for users to inspect the numerical and graphical data and draw their own conclusions. For example an upward trend in genotoxicity data that did not cross the threshold might still distinguish two compounds. The decision thresholds were set as follows:

The cytotoxicity threshold is set at 80% of the cell density reached by the untreated control cells. This is greater than 3 times the standard deviation of the background. A positive cytotoxicity result (+) is concluded if 1 or 2 compound dilutions produce a final cell density lower than the 80% threshold. A strong positive cytotoxicity result positive (++) is concluded when either (i) three or more compound dilutions produce a final cell density lower than the 80% threshold or (ii) at least one compound dilution produces a final cell density lower than a 60% threshold. A negative result (−) is concluded when no compound dilutions produce a final cell density lower than the 80% threshold. The lowest effective concentration (LEC) is the lowest test compound concentration that produces a final cell density below the 80% threshold.

The compound absorbance control allows a warning to be generated if a test compound is significantly absorbing. If the ratio of the absorbance of the compound control well to a well filled with media alone is >2, there is a risk of interference with interpretation. The cytotoxicity controls indicate that the cell lines are behaving normally. The ‘high’ MMS standard should reduce the final cell density to below the 80% threshold, and should be a lower value than the ‘low’ standard.

The genotoxic threshold is set at a relative GLuc induction of 1.8 (i.e. an 80% increase). This decision threshold is set at greater than 3 times the standard deviation of the background. A positive genotoxicity result (+) is concluded if a compound dilution produces a relative GLuc induction greater than the 1.8 threshold. Within the field of genetic toxicology it is occasionally desirable to assess assay results in a way that acknowledges variations in potency of genotoxic effect between different compounds. Hence, GLuc inductions may also be assessed using the following criterion: a strong positive genotoxicity result (++) is concluded if three or more compound dilutions produce a relative GLuc induction greater than the 1.8 threshold. A negative genotoxicity result (−) is concluded where no compound dilutions produce a relative GLuc induction greater than the 1.8 threshold. The LEC is the lowest test compound concentration that produces a relative GLuc induction greater than the 1.8 threshold. The genotoxic controls demonstrate that the cell lines are responding normally to DNA damage. The ‘high’ control must produce a luminescence induction >2, and be a greater value than the ‘low’ control. Anomalous brightness data is generated when the toxicity leads to a final cell density less than 30% that of the blank. Genotoxicity data is not calculated above this toxicity threshold. Compounds that tested negative for genotoxicity, were re-tested up to 10 mM or 5000 μg/ml, or to the limit of solubility or cytotoxicity.

The compound luminescence control allows a warning to be generated when a compound is highly auto-luminescent. If the ratio of the luminescence of the compound control well to the average luminescence from the wells filled with vehicle-treated GLuc-T01 cells is >0.05, there is a risk of interference with interpretation.

### Example 3

### pEP-GD532-GLuc Data

### Introduction

GFP has proved a very successful reporter for the GreenScreen HC genotoxicity assay. However GFP has a number of limitations that have instigated the search for alternative reporters.

Against this background, the inventors wished to develop a genotoxicity assay in which a luciferase was used as a reporter protein. Luciferases are enzymes that catalyse light producing chemical reactions. The light produced can be measured using an assay, and (under correct assay conditions) can be considered to be a direct measure of the amount of luciferase present. Therefore, the amount of light produced by a cell having a “GADD45α-luciferase” expression cassette is a measure of the activity of the GADD45α reporter elements, which in turn is a measure of the genotoxicity of the test compound.

### Which Luciferase

Of the array of luciferases available, the inventors chose to study Firefly luciferase (FLuc) and Gaussia luciferase (GLuc) for incorporation into the assay. FLuc was chosen as it is the best described of all luciferases with extensive literature available for the design of FLuc based assays. GLuc was chosen as, unlike FLuc, it is secreted from the cell.

FLuc was originally cloned from the firefly Photinus pyralis. FLuc catalyses the oxidation of luciferin in a chemical reaction producing light. Magnesium is required as co-factor in the reaction.

luciferin+ATP+O₂→oxyluciferin+AMP+light

FLuc has the highest described quantum yield (>88%) of all luciferases. The light output of the reaction peaks at 562 nm which is in the yellow-green portion of the spectrum. The half-life of the FLuc reaction is <10 minutes whilst the half life of the luciferase protein is generally accepted as ˜3 hours although other higher figures have been reported. A number of different reagents can be added to FLuc reactions to lengthen the half-life of the reaction such a Coenzyme A and certain cytidine nucleotides. The native FLuc protein is sequestered in the peroxisome of cells but mutants have been produced that can localise to the cytoplasm. If luciferin is added to cells expressing FLuc, very little light output is observed in live cells compared to when the cells are lysed.

Gaussia luciferase (GLuc) has been cloned from the marine copepod Gaussia princeps. GLuc catalyses the oxidation of coelenterazine in a luminescent reaction.

coelenterazine+O₂→coelenteramide+light

The light output of the reaction peaks at ˜470 nm which is in the blue portion of the spectrum whilst the half-life of the GLuc reaction is less than 30 seconds. The GLuc protein is naturally secreted and in cells expressing GLuc the vast majority of the protein is found in the extracellular environment. The GLuc protein has been reported to be stable and resistant to pH and temperature induced degradation.

### FLuc Reagents and Assay Method

The reagents for the FLuc assay were originally taken from the article Wettey FR, Jackson AP. Luciferase Reporter Assay. In: Reviews and Protocols in DT40 Research. Springer Netherlands, 2006, pp. 423-425. The reagents and their concentrations are listed below. The pH of both mixes is adjusted to pH 7.8 in order to maximise the light output from luciferase.

It was decided to directly lyse the TK6 cells in the GreenScreen HC assay medium as to pellet and wash the cells would add additional steps to the protocol and therefore increase variability into the data. Experimentally it was determined that the lysis and assay mix could be combined and added to the cells simultaneously. It was apparent that lysis occurs rapidly enough for immediate FLuc quantification. The solution of this combined lysis and assay mix (L&A buffer) is shown below.

L&A Buffer

| | CAS | | | | |
|---|---|---|---|---|---|
| Reagent | Number | Final Conc. | | Initial Conc. | |
| Tricine | 5704-04-1 | 20 | mM | 80 | mM |
| EDTA | 6381-92-6 | 0.1 | mM | 0.4 | mM |
| CDTA | 482-54-2 | 2 | mM | 8 | mM |
| (MgCO₃)₄*Mg(OH)₂*5H₂O | 56378-72-4 | 1.07 | mM | 4.28 | mM |
| MgSO₄*7H₂0 | 10034-99-8 | 2.67 | mM | 10.68 | mM |
| Glycerol | 56-81-5 | 10% | | 40% | |
| Triton X-100 | 9002-93-1 | 1% | | 4% | |
| Dithiothreitol | 3483-12-3 | 33.5 | mM | 134 | mM |
| Coenzyme A | 85-61-0 | 270 | μM | 1.08 | mM |
| ATP | 987-65-5 | 530 | μM | 2.12 | mM |
| Luciferin | 2591-17-5 | 470 | μM | 1.88 | mM |

The final concentration listed above is the concentration of the reagents after the L&A buffer has been combined with the GreenScreen HC assay media. The final concentration of the reagents is fixed and based on the information from the Wettey and Jackson protocol. The FLuc assay as it is currently performed relies on the addition of 40 μl of L&A buffer to 120 μl of GreenScreen HC assay buffer. Therefore, the initial concentration of the reagents in the L&A buffer has to be four times greater than the desired final concentration. The L&A buffer should be pH8.0 as when the L&A buffer is combined with the GreenScreen HC assay medium (pH7.2) this give a final pH of ˜7.8.

### GLuc Reagents and Assay Method

The preparation of the GLuc assay buffer is shown below. The complete assay buffer is incubated in the dark at room temperature for 20 minutes before being combined with an equal volume of GLuc sample. Coelenterazine spontaneously decays and is unstable for prolonged periods in aqueous solutions. Allowing coelenterazine to acclimatise to room temperature for 20 minutes will minimise variability in this spontaneous decay between samples.

GLuc Assay buffer

| | CAS | | |
|---|---|---|---|
| Reagent | Number | Volume | Concentration |
| NaCl (5M) | 7647-14-5 | 20 | 1M |
| Coelenterazine native (2 mg/ml) | 55779-48-1 | 0.847 | 40 μM |
| 2.5x GreenScreen HC media | — | 40 | 1x |
| H₂O | 7732-18-5 | 39.153 | — |

As mentioned in above, the assay can also be performed using a coelenterazine solution buffered to pH 7.4. Here the coelenterazine is prepared as a 5 mM stock solution in acidified methanol. A Luminescence Buffer is prepared (400 mM Tris-HCl; 5 mM β-Cyclodextrin; Deionised water; buffered to pH 7.4 with 10 N NaOH). The stock coelenterazine solution is then diluted 2000-fold in the luminescence buffer to give 2.5 μM coelenterazine solution buffered to pH to 7.4 by TRIS). This is the injection solution which is added to the reaction assay (leading to a further 4-fold dilution of coelenterazine).

### Results—Comparison of GLuc to FLuc and GFP

The preparation of pEP-GD532-GLuc is described in the accompanying examples. Using a similar strategy the inventors also prepared plasmid pEP-GD532-L, in which FLuc is used as the reporter protein. TK6 cells are transfected with a plasmid having a GD532-L or GD532-GLuc expression cassette by electroporation and clones bearing the reporter plasmids are selected.

The inventors wished to determine which of the GLuc and FLuc reporter proteins were most suitable for use in a genotoxicity assay. To determine this, they performed a series of experiments in which cells having the GD532-L or GD532-GLuc expression cassette were exposed to a test compound, and the activity of GLuc and FLuc measured and compared to the standard GADD45α-GFP expression cassette.

### Effect of MNU

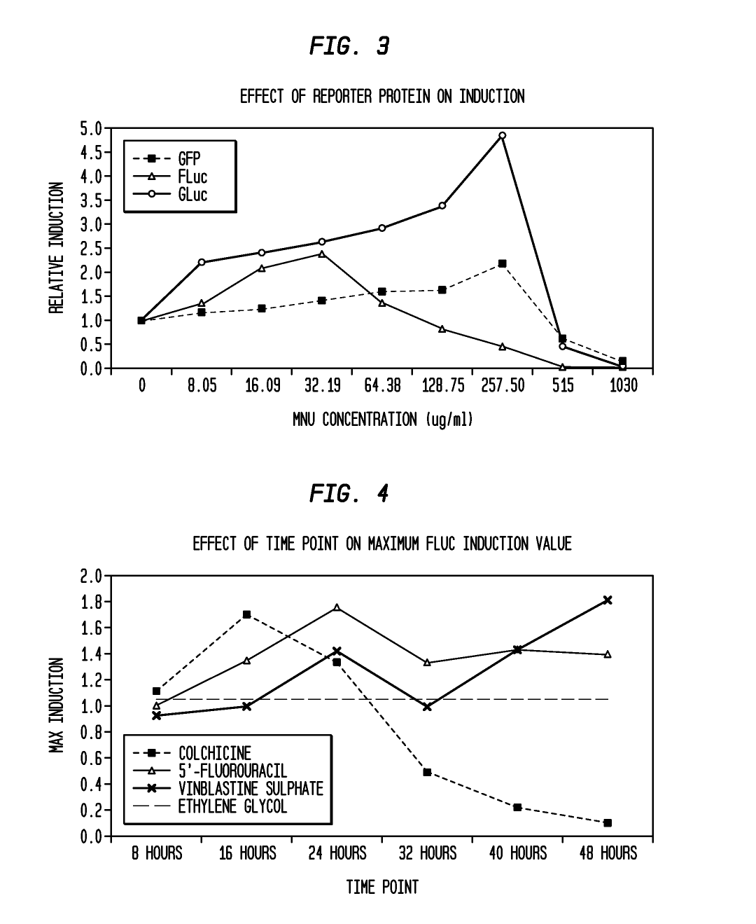

The data presented in FIG. 3 shows how methyl-nitrosurea (MNU) causes GADD45α induction, as reported by GFP, FLuc and GLuc. Studying FIG. 3 allows for the construction of a number of hypotheses regarding the stability of the reporter proteins and how this will affect the GreenScreen HC assay. The FLuc protein has been reported to have a half-life within cells of ˜3 hours. In comparison, GFP has been reported to have a half-life within cells of ˜26 hours. In this respect GFP can be considered to give more of a cumulative measure of GADD45α induction whilst FLuc will report only on recent GADD45 induction.

GADD45α induction does not peak until at least 258 μg/ml of MNU as demonstrated by the peak in GFP signal at this concentration. MNU concentrations greater than 32 μg/ml cause significant cell death which explains the decrease in FLuc signal at higher concentration of MNU. At the two highest concentrations of MNU there is little detectable FLuc signal, as all cells have died early in the experimental time course and any protein produced has since been degraded. In contrast there are clearly detectable levels of both GFP and GLuc at the two highest MNU concentrations demonstrating that these two proteins have higher stability than FLuc. It should be noted that GLuc differs from both FLuc and GFP in that the protein is secreted from the cell. This means that when the assay is set up, the vast majority of GLuc protein is separated from the TK6 cells as they are washed in PBS and GreenScreen HC assay medium. The GFP and FLuc proteins are cytoplasmic and therefore are present in significant quantity at the start of the assay. FIG. 3 also shows the different compound test concentrations at which the highest relative induction is seen—this is lowest for FLuc (32 μg/ml), and reflects the loss of FLuc signal in dead and dying cells. Furthermore, the magnitude of measurable GADD45α response is clearly far greater for GLuc for MNU-treated cells, when compared with GFP and FLuc.

Taken as a whole, the data presented in FIG. 3 shows that GFP and GLuc both accumulate, whilst FLuc does not. The FLuc induction peak appears to be at a lower test compound concentration, and the signal drops away at higher concentrations (at these higher concentrations the test compound is very toxic within the 24 hour timeframe of the assay. Effectively, as cells experience toxicity/die, the FLuc signal dies with them. This is because FLuc is an unstable protein with a short half-life that also has energetic requirements. GFP has a much longer half-life than FLuc and hence accumulates and persists even when cells are in toxic conditions and dying, hence the peak of induction can be at much higher concentrations. Importantly, GLuc is also a relatively stable protein and also illustrates accumulation similar to GFP. This is an important advantage for using GLuc as a luciferarase reporter protein rather than FLuc. If FLuc were used, it would be necessary to measure data at number of time points to ensure that the luciferase signal was a measure of GADD45α activity, and hence the genotoxicity of the test compound, rather than the response affected significantly by reporter protein degradation and cytotoxic effects of the test agent.

### Effect of 4 Test Compounds on FLuc Activity

FLuc cells were combined with a several known genotoxins and non-genotoxins. FLuc expression was measured at 8, 16, 24, 32, 40 and 48 hours after treatment. The results shown revealed that for the 4 compounds tested, maximum induction was observed at either 16, 24 or 48 hours after treatment. FIG. 4 shows the maximum induction values over the time course for three genotoxins (Colchicine, 5-Fluorouracil and Vinblastine sulphate) and one non-toxic non-genotoxin (ethylene glycol). FIG. 4 demonstrates that the maximum GLuc induction was achieved at different timepoints for different test compounds. Colchicine and probably 5-fluorouracil would not have been detected as genotoxic using the 48 hour endpoint preferred in the GreenScreen HC assay. This means that a number of time points would be required to detect all known genotoxins and this implies that a usable assay would need to be performed kinetically. However, this result is problematic as FLuc induction determination requires the cells to be lysed, precluding multiple timepoints in individual cells. Furthermore, the same compounds illustrated in FIG. 4 were correctly identified (3 genotoxins and 1 non-genotoxin) in an assay using a 48 hour endpoint with GLuc as the reporter protein.

Therefore FIG. 4 demonstrates the measurement timepoint problem for FLuc, due to the protein instability and lack of accumulation.

### Summary

The disadvantage of the short FLuc half-life is that a genotoxicity assay using FLuc will require multiple time points (three or more) to ensure that the peak FLuc induction is recorded. This is a significant problem as cell lysis is required to determine FLuc concentration; parallel assay microplates would have to be set up for each time point. GLuc offers at least two advantages over FLuc. First, GLuc is secreted so its presence can be determined without cell lysis. Secondly, GLuc is more stable than FLuc which might preclude the need for more than one time point.

From this data the inventors concluded that GLuc has better characteristics than FLuc for use as a luciferase reporter protein in a genotoxicity assay.

Results—Genotoxicity Data Using pEP-GD532-GLuc

A series of genotoxicity assays were performed using a TK6 cell line having the GD532-GLuc expression cassette (a “GLuc assay”). The assays were performed using the experimental protocol provided in a later example.

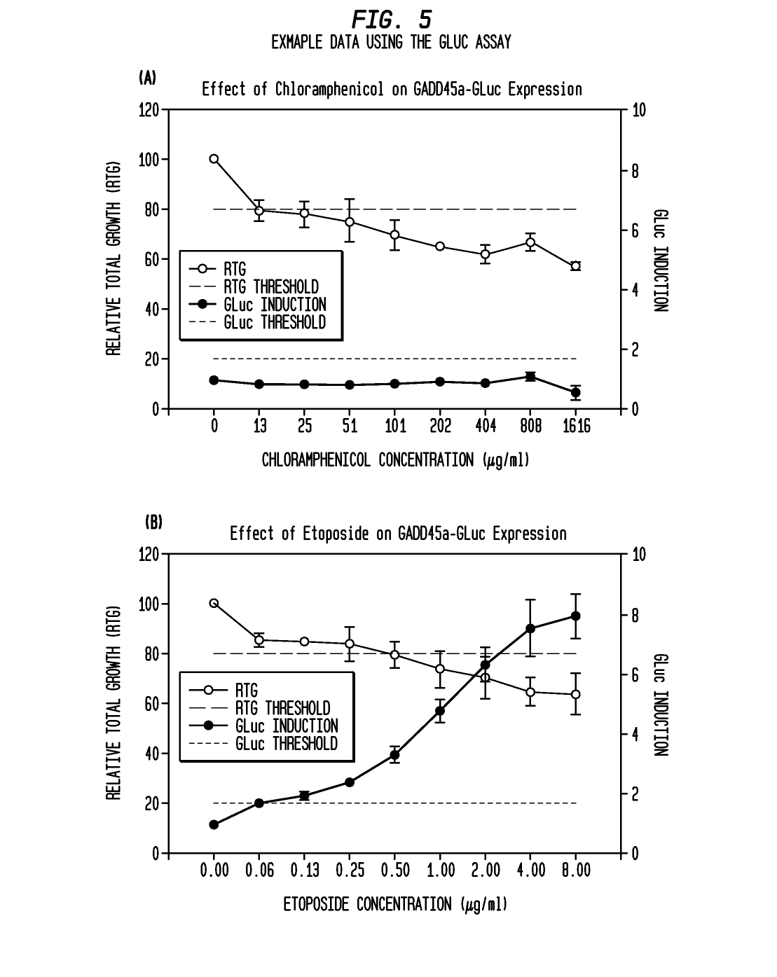

Example data from the assays are provided in FIG. 5. Here it can be seen in panel A that Chloramphenicol, a non-genotoxin, was detected as negative as expected in the GLuc assay. In contrast, in panel B the genotoxin Etoposide is detected as positive as expected in the GLuc assay.

We also include below a list of example genotoxicity results for different classes of genotoxin tested with the GD532-GLuc reporter system.

| | | GLuc Genotox | |
|---|---|---|---|
| Compound | CAS No. | Result | LEC/ug/ml |
| Direct-acting | | | |
| Cisplatin | 15663-27-1 | +++ | 0.25 |
| Mitomycin C | 50-07-7 | +++ | 0.13 |
| Methyl methanesulfonate | 66-27-3 | +++ | 6.25 |
| N-Methyl-N-nitro-nitosoguanidine | 70-25-7 | +++ | 0.39 |
| N-Nitroso-N-methylurea | 684-93-5 | +++ | 8.05 |
| 4-Nitroquinoline-1-oxide | 56-57-5 | +++ | 0.13 |
| Topoisomerase inhibitors | | | |
| Camptothecin | 7689-03-4 | +++ | 0.08 |
| Etoposide | 33419-42-0 | +++ | 0.06 |
| Aneugens | | | |
| Benomyl | 17804-35-2 | +++ | 1.81 |
| Griseofulvin | 126-07-8 | +++ | 5.50 |
| Paclitaxel (Taxol) | 33069-62-4 | +++ | 0.03 |
| Vincristine Sulphate | 2068-78-2 | +++ | 0.0008 |
| Nucleotide/DNA synthesis | | | |
| inhibitors | | | |
| 5-Azacytidine | 320-67-2 | +++ | 0.38 |
| 5-Fluorouracul | 51-21-8 | +++ | 0.63 |
| Aphidicolin | 38966-21-1 | +++ | 0.13 |
| Hydroxyurea | 127-07-1 | +++ | 4.75 |
| Pyrimethamine | 58-14-0 | +++ | 0.39 |
| Reactive oxygen species | | | |
| Hydrogen Peroxide | 7722-84-1 | +++ | 5.00 |

Each ‘+’ represents the outcome in an individual assay, i.e. the test compounds were all tested in triplicate. All test compounds listed were positively identified as genotoxic agents by the GD532-GLuc reporter system

### Results—High Signal to Noise Ratio and Luminescent Output Reduces the Impact of Fluorescent Interference

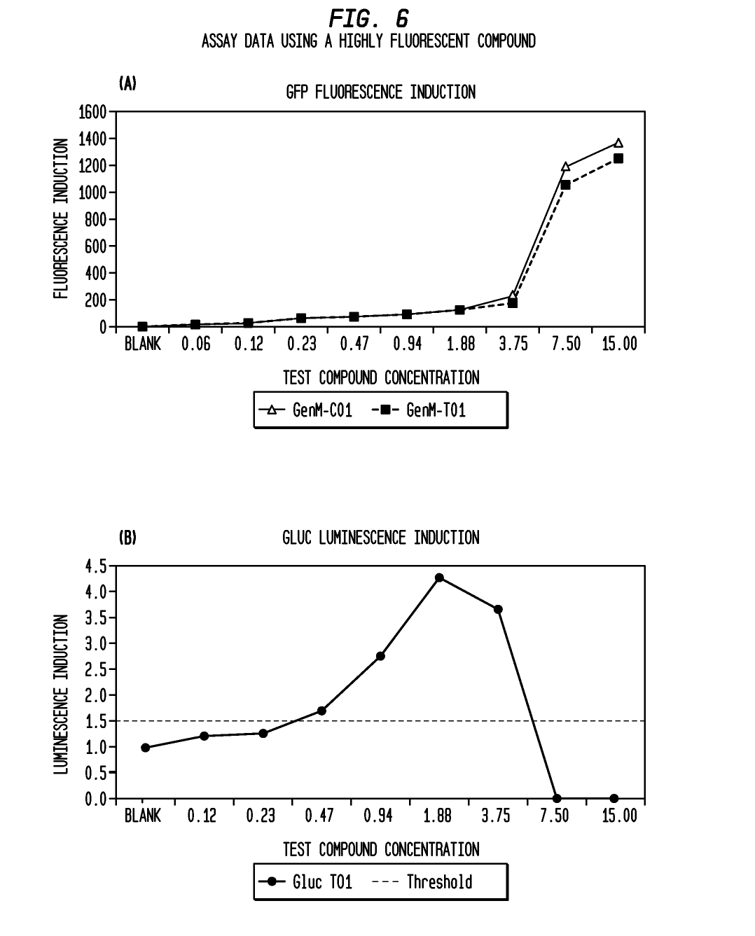

To further characterise the genotoxicity assay using the GLuc reporter protein, the inventors assessed the “signal to noise” ratio of an assay of a highly fluorescent test compound using GLuc and GFP reporter proteins. The data generated can be seen in FIG. 6. Note that there is little or no separation between the fluorescent strain (lower line) and non-fluorescent strain (upper line) in panel (A). This is due to the autofluorescence from the compound which effectively masks the fluorescence from the GFP reporter protein. In contrast there is a clear positive signal from the GLuc system without any interference.

Here it can be seen that an assay using GLuc as a reporter protein generates a high intensity light output with a background of approximately zero. An advantage of using luminescence as a reporter assay is that there is no need for incident light, as used in fluorescence based assays. This means that there is no excitation of unwanted fluorescence which would mask the signal from the GFP reporter protein. By using GLuc rather than GFP, even highly fluorescent compounds can be tested without causing a problem for the GLuc output. As a consequence luciferase measurement is less likely to suffer interference from coloured or fluorescent test materials.

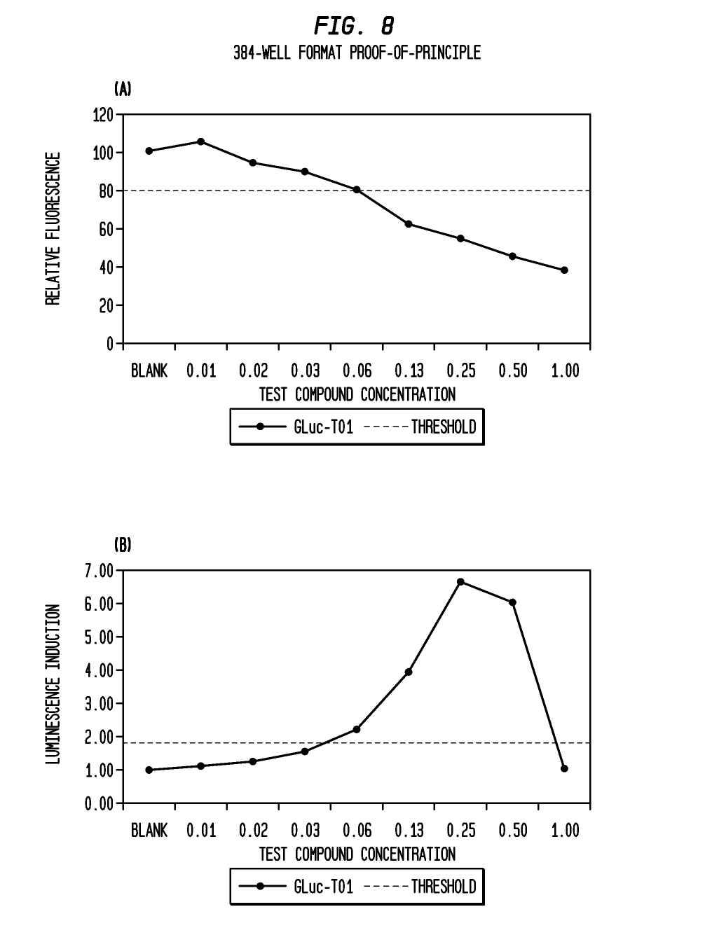

Additionally, the high ‘signal to noise’ ratio allows genotoxicity assays using GLuc-mediated bioluminescence to be conducted using 384-well microtitre plates, as can be seen from the data presented in FIG. 8.

### Example 4

### An Adapted Genotoxicity Assay Using GLuc for Metabolic Activation Studies

The inventors have adapted the genotoxicity assay described above and in the accompanying examples to allow the of S9 liver extracts into the assay. By using S9 extracts, the assay permits the detection of pro-mutagens or pro-genotoxins—compounds that are not inherently genotoxic in their native form but can become so due to metabolic reactions.

S9 is a liver extract (known to the skilled person) that allows for the detection of those compounds that are non-genotoxic in their native forms but that may be chemically altered by metabolism (primarily in the liver) to generate a genotoxic compound in vivo.

S9 extract can be incorporated into an adaptation of the assay method outlined in Example 2 above, either in a parallel assay to the method in Example 2 or as an independent assay. In an S9-incorporating assay, GLuc-T01 cells are exposed to the test compound in the presence of the S9 extract in a mixture with enzyme co-factors (for example, glucose-6-phosphate (2.5 mM) and β-nicotinamide adenine dinucleotide phosphate (0.5 mM)). The S9 extract is normally used at a final concentration of 1% (v/v) in the assay microplate. The incubation time with test compounds and S9 mix is generally 3 hours before the S9 and test compound are removed, cells washed in PBS and then resuspended in fresh assay medium for the remaining 45 hours of incubation. The conditions of an S9-incorporating assay (for example, time of exposure and type of S9-animal species, chemical induction of hepatic enzymes etc.) may be varied according to experimental requirements.

### Adapted Protocol

Use of the plate reader measurements were found to result in reduced sensitivity of the assay for pro-genotoxins in S9 metabolic activation studies. This was unexpected. The inventors investigated this matter further, and found that this was due to the relative insensitivity of the optical absorbance measurement used to estimate cell density and for normalisation of the reporter output. The relative insensitivity meant that some of the typical standard pro-genotoxic compounds were not reliably detected as genotoxic.

The inventors subsequently discovered that a fluorescent cell stain could be used to replace the optical absorbance measure. This is because the two methods are effectively different ways of estimating the same thing. Surprisingly, the method by which the cell stain was used improved the sensitivity of cell number estimation and hence the detection of pro-genotoxins.

The cell stain used in the adapted protocol is thiazole orange (TO) which is a cyanine dye that binds to nucleic acids. The binding of TO to DNA and RNA greatly enhances its fluorescence intensity, allowing for its detection without the need to wash away background, unbound TO.

The method requires GLuc-T01 cells to be lysed to allow access to the DNA of all cells present in the microplate well. The amount of nucleic acid present is proportional to the number of cells and hence the fluorescence intensity from DNA-bound TO is also proportional to the number of cells.

TO is dissolved in 100% DMSO to form a stock solution at 25 mM. This is mixed with a cell lysis solution consisting of PBS and Triton-X100. 50 μl of the TO/lysis mix are added to each microplate well and incubated for between 5 and 20 minutes prior to taking fluorescence measurements (485 nm excitation and 535 nm emission). In the microplate, the final concentration of TO is 15 μM and for Triton-X100 it is 1% (v/v).

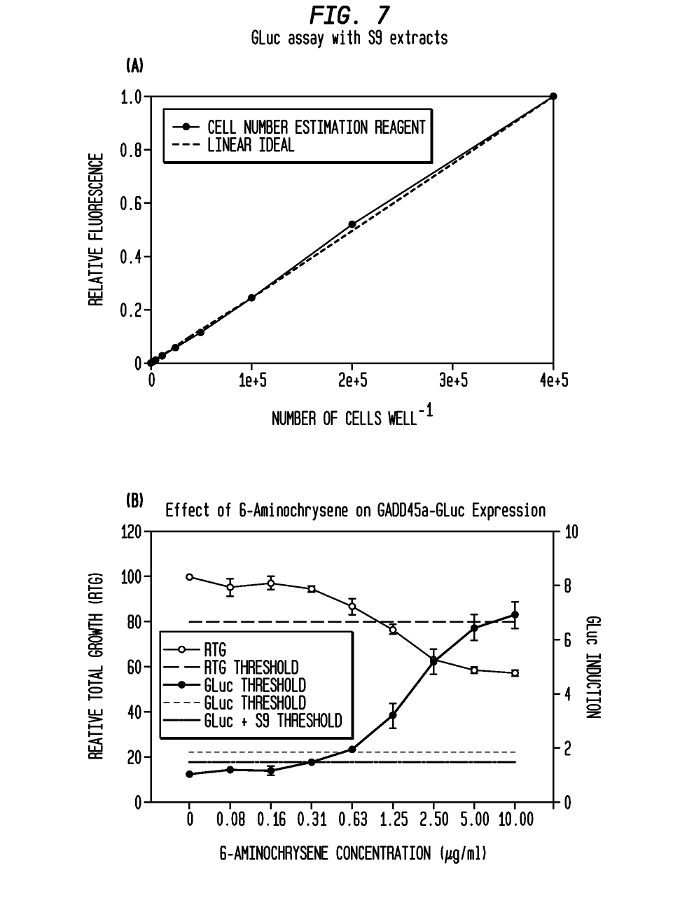

FIG. 7 shows a Calibration of the TO fluorescence with cell number (using optimised conditions and cell densities relevant to the assay) (A) and example data for a standard pro-genotoxin (6-Aminochrysene) detected using the S9 metabolic activation GLuc assay, incorporating the TO cell number estimation (B).

As discussed in Example 2 above, it is useful to have clear definitions of positive and negative results from routine assays and such definitions have been derived, taking into account the maximum noise in the system and data from chemicals where there is a clear consensus on genotoxicity and mechanism of action.

Where the assay includes S9 liver extracts, the genotoxic threshold is set at a relative GLuc induction of 1.5 (i.e. an 50% increase). Hence a positive genotoxicity result (+) is concluded if a test compound produces a relative GLuc induction greater than the 1.5 threshold.

| Sequence of pEP-GD532 GLuc (SEQ ID NO: 1) | |
|---|---|
| 1 gtcgaccaat tctcatgttt gacagcttat catcgcagat ccgggcaacg | |
| 51 ttgttgccat tgctgcaggc gcagaactgg taggtatgga agatcttggg | |
| 101 tggggcactt taggactgtg gttcatttga attggtgtaa acaatacacc | |
| 151 ggttctactg tcctacagcc tccattcaga tgactgaagt catgggactt | |
| 201 tcagcatagc tagctgatga cagtgcatac tattttgtcc caaaatccag | |
| 251 ttcaagcatg gacataccaa taagagccta agctctttaa aggcaaagga | |
| 301 ccaggaattg tacagttctt ggtatagaag aagacaggca aaagtgtttt | |
| 351 tgaactaacg ttaaatgtgc aatatgttag aattcatgca atgcacagga | |
| 401 ctgcaggatt ctgatatctt atttaactct caaattctat tcaactcaat | |
| 451 aaaccttgac tgtgcttcta ctaaatgcag gtattgtact aggagctgag | |
| 501 gacaccaaac tgatgaagtc cttgctgtca agaaactcac atgattccct | |
| 551 aattctttgt cagcttgctg tgatcacatt ttcttcccaa gaacctctaa | |
| 601 gaaatgccta gtggatagaa ccttggagtt ccacggaaca tattaacaat | |
| 651 cgccaaatga tgactcaggc tagattgtgt aattcaggtt ttgtctgcaa | |
| 701 aactgaaaat gcttcggtaa cctacctaaa tttcaatgtt gaggaattct | |
| 751 ttaagaaaga catcaaatgt taagatttaa ggcatagata tgagatacat | |
| 801 agtcatgctt aggtgaatta tgcactgacc atgaccattt ctttactcaa | |
| 851 atgttgtcca tggctgacaa cacagtgaaa aaatgagtgc aaaatgacaa | |
| 901 ctcaaataaa tgaaccagaa aacctatcac ttttcttttc caccaaatta | |
| 951 agatcaagag agctggagaa tattttgtct agagtgataa aaacataagg | |
| 1001 gtgcaaaact tccaggtacc tttgcagaaa ttacttctgt gacctttggc | |
| 1051 tgtacagcaa ccttaataat gcaagcactg ttttgaatgc aagcatgtgg | |
| 1101 gagccatttt caccactttt gatgacttca gtaggtttaa gaaatgtttt | |
| 1151 tgcttttatt gcataaacca taaaacaaag gaagggactt ttgaactact | |
| 1201 cagtgagagt ctatatatta aagtttgttt ttcaaaaatg tgtaactacc | |
| 1251 atttgcagtt ttaaaggtct gctttccacc tacaagttgc cattatctca | |
| 1301 aaggtgaaat tttagcatat gactaaaaac ttcctatagt tacagcttca | |
| 1351 tgattcagca tctaacatca ataattcaca gtgagatcat aggaggctct | |
| 1401 ctgtggaagg taacgacata catacgttag gaaaggaagc ttagggcata | |
| 1451 tcgagagcat tttgaattta gacttgtggg ctgtgtgggt gtcagatggt | |
| 1501 tgtctctcag ctggtgggcg tccagaagga tccttgtttg ggcaaggctc | |
| 1551 tttgagaaag gagaatctgg gttgccaggg attcccacat gtggtcacca | |
| 1601 gctccccacg cagaccagct cacgatttcc cagttacacc gggcaggtgg | |
| 1651 gaaaccgttc tgctttctgt ggaaaagatt ctaacttggt tccctgccat | |
| 1701 ccctgaatac aaacgggttg gtttttcttt tttgagcttc caacccttgc | |
| 1751 agctttccaa aaataaatca aaccagccat cagggcaccg aaataatact | |
| 1801 actgctaata agcagcttcg cctagactta gataaacaac acttctgagg | |
| 1851 taaactttgc cccggaggtc tggagacact tttttaatgt aacctgctta | |
| 1901 ctaataatta ctagacttca gtgcattaac cctggaaata gattttaata | |
| 1951 gccacccctt aaaacaaaag acatgaaaag ataataagaa aaaagtgccg | |
| 2001 caactattat agaaaaacac ttggcagcct gcttcagccc aagctgaggc | |
| 2051 cacctctagc ctctgctaaa gccccccact cccaatggtc cccgccaacc | |
| 2101 ggataagagt gcgcgcggga cccgccttcc cctctcggca ccgcccccgc | |
| 2151 ccccgccccc tcggctcgcc tcccgcgtgg ctcctccctt ttccgctcct | |
| 2201 ctcaacctga ctccaggagc tggggtcaaa ttgctggagc aggctgattt | |
| 2251 gcatagccca atggccaagc tgcatgcaaa tgaggcggaa ggtggttggc | |
| 2301 tgagggttgg caggataacc ccggagagcg gggccctttg tcctccagtg | |
| 2351 gctggtaggc agtggctggg aggcagcggc ccaattagtg tcgtgcggcc | |
| 2401 cgtggcgagg cgaggtccgg ggagcgagcg agcaagcaag gcgggagggg | |
| 2451 tggccggagc tgcggcggct ggcacaggag gaggagcccg ggcgggcgag | |
| 2501 gggcggccgg agagcgccag ggcctgagct gccggagcgg cgcctgtgag | |
| 2551 tgagtgcaga aagcaggcgc ccgcgcgcta gccgtggcag gagcagcccg | |
| 2601 cacgccgcgc tctctccctg ggcgacctgc agtttgcaat atgggagtca | |
| 2651 aagttctgtt tgccctgatc tgcatcgctg tggccgaggc caagcccacc | |
| 2701 gagaacaacg aagacttcaa catcgtggcc gtggccagca acttcgcgac | |
| 2751 cacggatctc gatgctgacc gcgggaagtt gcccggcaag aagctgccgc | |
| 2801 tggaggtgct caaagagatg gaagccaatg cccggaaagc tggctgcacc | |
| 2851 aggggctgtc tgatctgcct gtcccacatc aagtgcacgc ccaagatgaa | |
| 2901 gaagttcatc ccaggacgct gccacaccta cgaaggcgac aaagagtccg | |
| 2951 cacagggcgg cataggcgag gcgatcgtcg acattcctga gattcctggg | |
| 3001 ttcaaggact tggagcccat ggagcagttc atcgcacagg tcgatctgtg | |
| 3051 tgtggactgc acaactggct gcctcaaagg gcttgccaac gtgcagtgtt | |
| 3101 ctgacctgct caagaagtgg ctgccgcaac gctgtgcgac ctttgccagc | |
| 3151 aagatccagg gccaggtgga caagatcaag ggggccggtg gtgactaatg | |
| 3201 cggccgcgac tctagatcat aatcagccat accacatttg tagaggtttt | |
| 3251 acttgcttta aaaaacctcc cacacctccc cctgaacctg aaacataaaa | |
| 3301 tgaatgcaat tgttgttgtt atcgcgaccc cgataacgtg gtgttgtgcc | |
| 3351 tgctggcggc ggacgaggac gacgacagag atgtggctct gcagatccac | |
| 3401 ttcaccctga tccaggcgtt ttgctgcgag aacgacatca acatcctgcg | |
| 3451 cgtcagcaac ccgggccggc tggcggagct cctgctcttg gagaccgacg | |
| 3501 ctggccccgc ggcgagcgag ggcgccgagc agcccccgga cctgcactgc | |
| 3551 gtgctggtga cggtaaggga ctgggggact gcagcctgca gggtagagcc | |
| 3601 ccggaaggac gggagtcagg actgggttgc ctgattgtgg atctgtggta | |
| 3651 ggtgagggtc aggagggtgg ctgcctttgc ccgactagag tgtggctgga | |
| 3701 ctttcagccg agatgtgcta gtttcatcat caggattttc tgtggtacag | |
| 3751 aacatgtcta agcatgctgg ggactgccag cagcggaaga gatccctgtg | |
| 3801 agtcagcagt cagcccagct actccctacc tacatctgca ctgcctcccg | |
| 3851 tgactaattc ctttagcagg gcagattaga taaagccaaa tgaattcctg | |
| 3901 gctcacccct cattaaggag tcagcttcat tctctgccag tcagagctaa | |
| 3951 aaatagaaat tgtgtaggag acaaaccttg ttaattccct agaaatacat | |
| 4001 taagaggata gagtggaatt ttttttctct gcaatcttgc atttttttaa | |
| 4051 tggctctttt tttttttcct gataaaaacc tttggtaggt agggaagtta | |
| 4101 tgttttcagg ggtaaatgtg ctacttttgt cttctaaatt ttgctctttt | |
| 4151 ttgactggtc tagtcaagtg acagcccgat tattttgcta ctccttaaaa | |
| 4201 gtactattct gtctcttgga gtatggttga tggcaattcc agttaactgc | |
| 4251 tgtgcagctc tcatctcatt gtgcacacag catggaaatc tttctcaaaa | |
| 4301 ctgtttcact caggtcaggg taacaagttt ggtagagcaa accggtgaat | |
| 4351 gatactctca tgcaaaactg aacagatatg caaacatatg tatgtggttc | |
| 4401 agcttgggtt gcatgggttc agactttgca atgtgtagtt taataggtaa | |
| 4451 ttacccttaa cgcttttgca gggaacccaa ctaccttgaa gaaactttaa | |
| 4501 tttttttgtg cttctaattt gtctccatgt cacatagcca aaatatagaa | |
| 4551 tgttcaagtg ttttctcctc aaaagtataa ttactagaat atactggttt | |
| 4601 ttaaaataag tttattttta taaatttgtt tccagaatcc acattcatct | |
| 4651 caatggaagg atcctgcctt aagtcaactt atttgttttt gccgggaaag | |
| 4701 tcgctacatg gatcaatggg ttccagtgat taatctccct gaacggtgat | |
| 4751 ggcatctgaa tgaaaataac tgaaccaaat tgcactgaag tttttgaaat | |
| 4801 acctttgtag ttactcaagc agttactccc tacactgatg caaggattac | |
| 4851 agaaactgat gccaaggggc tgagtgagtt caactacatg ttctgggggc | |
| 4901 ccggagatag atgactttgc agatggaaag aggtgaaaat gaagaaggaa | |
| 4951 gctgtgttga aacagaaaaa taagtcaaaa ggaacaaaaa ttacaaagaa | |
| 5001 ccatgcagga aggaaaacta tgtattaatt tagaatggtt gagttacatt | |
| 5051 aaaataaacc aaatatgtta aagtttaagt gtgcagccat agtttgggta | |
| 5101 tttttggttt atatgccctc aagtaaaaga aaagccgaaa gggttaatca | |
| 5151 tatttgaaaa ccatatttta ttgtattttg atgagatatt aaattctcaa | |
| 5201 agttttatta taaattctac taagttattt tatgacatga aaagttattt | |
| 5251 atgctataaa ttttttgaaa cacaatacct acaataaact ggtatgaata | |
| 5301 attgcatcat ttcttattgt gtgctcgagg ccggcaaggc cggatccaga | |
| 5351 catgataaga tacattgatg agtttggaca aaccacaact agaatgcagt | |
| 5401 gaaaaaaatg ctttatttgt gaaatttgtg atgctattgc tttatttgta | |
| 5451 accattataa gctgcaataa acaagttaac aacaacaatt gcattcattt | |
| 5501 tatgtttcag gttcaggggg aggtgtggga ggttttttaa agcaagtaaa | |
| 5551 acctctacaa atgtggtatg gctgattatg atccggctgc ctcgcgcgtt | |
| 5601 tcggtgatga cggtgaaaac ctctgacaca tgcagctccc ggagacggtc | |
| 5651 acagcttgtc tgtaagcgga tgccgggagc agacaagccc gtcagggcgc | |
| 5701 gtcagcgggt gttggcgggt gtcggggcgc agccatgagg tcgactctag | |
| 5751 aggatcgatg ccccgccccg gacgaactaa acctgactac gacatctctg | |
| 5801 ccccttcttc gcggggcagt gcatgtaatc ccttcagttg gttggtacaa | |
| 5851 cttgccaact gggccctgtt ccacatgtga cacggggggg gaccaaacac | |
| 5901 aaaggggttc tctgactgta gttgacatcc ttataaatgg atgtgcacat | |
| 5951 ttgccaacac tgagtggctt tcatcctgga gcagactttg cagtctgtgg | |
| 6001 actgcaacac aacattgcct ttatgtgtaa ctcttggctg aagctcttac | |
| 6051 accaatgctg ggggacatgt acctcccagg ggcccaggaa gactacggga | |
| 6101 ggctacacca acgtcaatca gaggggcctg tgtagctacc gataagcgga | |
| 6151 ccctcaagag ggcattagca atagtgttta taaggccccc ttgttaaccc | |
| 6201 taaacgggta gcatatgctt cccgggtagt agtatatact atccagacta | |
| 6251 accctaattc aatagcatat gttacccaac gggaagcata tgctatcgaa | |
| 6301 ttagggttag taaaagggtc ctaaggaaca gcgatatctc ccaccccatg | |
| 6351 agctgtcacg gttttattta catggggtca ggattccacg agggtagtga | |
| 6401 accattttag tcacaagggc agtggctgaa gatcaaggag cgggcagtga | |
| 6451 actctcctga atcttcgcct gcttcttcat tctccttcgt ttagctaata | |
| 6501 gaataactgc tgagttgtga acagtaaggt gtatgtgagg tgctcgaaaa | |
| 6551 caaggtttca ggtgacgccc ccagaataaa atttggacgg ggggttcagt | |
| 6601 ggtggcattg tgctatgaca ccaatataac cctcacaaac cccttgggca | |
| 6651 ataaatacta gtgtaggaat gaaacattct gaatatcttt aacaatagaa | |
| 6701 atccatgggg tggggacaag ccgtaaagac tggatgtcca tctcacacga | |
| 6751 atttatggct atgggcaaca cataatccta gtgcaatatg atactggggt | |
| 6801 tattaagatg tgtcccaggc agggaccaag acaggtgaac catgttgtta | |
| 6851 cactctattt gtaacaaggg gaaagagagt ggacgccgac agcagcggac | |
| 6901 tccactggtt gtctctaaca cccccgaaaa ttaaacgggg ctccacgcca | |
| 6951 atggggccca taaacaaaga caagtggcca ctcttttttt tgaaattgtg | |
| 7001 gagtgggggc acgcgtcagc ccccacacgc cgccctgcgg ttttggactg | |
| 7051 taaaataagg gtgtaataac ttggctgatt gtaaccccgc taaccactgc | |
| 7101 ggtcaaacca cttgcccaca aaaccactaa tggcaccccg gggaatacct | |
| 7151 gcataagtag gtgggcgggc caagataggg gcgcgattgc tgcgatctgg | |
| 7201 aggacaaatt acacacactt gcgcctgagc gccaagcaca gggttgttgg | |
| 7251 tcctcatatt cacgaggtcg ctgagagcac ggtgggctaa tgttgccatg | |
| 7301 ggtagcatat actacccaaa tatctggata gcatatgcta tcctaatcta | |
| 7351 tatctgggta gcataggcta tcctaatcta tatctgggta gcatatgcta | |
| 7401 tcctaatcta tatctgggta gtatatgcta tcctaattta tatctgggta | |
| 7451 gcataggcta tcctaatcta tatctgggta gcatatgcta tcctaatcta | |
| 7501 tatctgggta gtatatgcta tcctaatctg tatccgggta gcatatgcta | |
| 7551 tcctaataga gattagggta gtatatgcta tcctaattta tatctgggta | |
| 7601 gcatatacta cccaaatatc tggatagcat atgctatcct aatctatatc | |
| 7651 tgggtagcat atgctatcct aatctatatc tgggtagcat aggctatcct | |
| 7701 aatctatatc tgggtagcat atgctatcct aatctatatc tgggtagtat | |
| 7751 atgctatcct aatttatatc tgggtagcat aggctatcct aatctatatc | |
| 7801 tgggtagcat atgctatcct aatctatatc tgggtagtat atgctatcct | |
| 7851 aatctgtatc cgggtagcat atgctatcct catgcatata cagtcagcat | |
| 7901 atgataccca gtagtagagt gggagtgcta tcctttgcat atgccgccac | |
| 7951 ctcccaaggg ggcgtgaatt ttcgctgctt gtccttttcc tgctggttgc | |
| 8001 tcccattctt aggtgaattt aaggaggcca ggctaaagcc gtcgcatgtc | |
| 8051 tgattgctca ccaggtaaat gtcgctaatg ttttccaacg cgagaaggtg | |
| 8101 ttgagcgcgg agctgagtga cgtgacaaca tgggtatgcc caattgcccc | |
| 8151 atgttgggag gacgaaaatg gtgacaagac agatggccag aaatacacca | |
| 8201 acagcacgca tgatgtctac tggggattta ttctttagtg cgggggaata | |
| 8251 cacggctttt aatacgattg agggcgtctc ctaacaagtt acatcactcc | |
| 8301 tgcccttcct caccctcatc tccatcacct ccttcatctc cgtcatctcc | |
| 8351 gtcatcaccc tccgcggcag ccccttccac cataggtgga aaccagggag | |
| 8401 gcaaatctac tccatcgtca aagctgcaca cagtcaccct gatattgcag | |
| 8451 gtaggagcgg gctttgtcat aacaaggtcc ttaatcgcat ccttcaaaac | |
| 8501 ctcagcaaat atatgagttt gtaaaaagac catgaaataa cagacaatgg | |
| 8551 actcccttag cgggccaggt tgtgggccgg gtccaggggc cattccaaag | |
| 8601 gggagacgac tcaatggtgt aagacgacat tgtggaatag caagggcagt | |
| 8651 tcctcgcctt aggttgtaaa gggaggtctt actacctcca tatacgaaca | |
| 8701 caccggcgac ccaagttcct tcgtcggtag tcctttctac gtgactccta | |
| 8751 gccaggagag ctcttaaacc ttctgcaatg ttctcaaatt tcgggttgga | |
| 8801 acctccttga ccacgatgct ttccaaacca ccctcctttt ttgcgcctgc | |
| 8851 ctccatcacc ctgaccccgg ggtccagtgc ttgggccttc tcctgggtca | |
| 8901 tctgcggggc cctgctctat cgctcccggg ggcacgtcag gctcaccatc | |
| 8951 tgggccacct tcttggtggt attcaaaata atcggcttcc cctacagggt | |
| 9001 ggaaaaatgg ccttctacct ggagggggcc tgcgcggtgg agacccggat | |
| 9051 gatgatgact gactactggg actcctgggc ctcttttctc cacgtccacg | |
| 9101 acctctcccc ctggctcttt cacgacttcc ccccctggct ctttcacgtc | |
| 9151 ctctaccccg gcggcctcca ctacctcctc gaccccggcc tccactacct | |
| 9201 cctcgacccc ggcctccact gcctcctcga ccccggcctc cacctcctgc | |
| 9251 tcctgcccct cctgctcctg cccctcctcc tgctcctgcc cctcctgccc | |
| 9301 ctcctgctcc tgcccctcct gcccctcctg ctcctgcccc tcctgcccct | |
| 9351 cctgctcctg cccctcctgc ccctcctcct gctcctgccc ctcctgcccc | |
| 9401 tcctcctgct cctgcccctc ctgcccctcc tgctcctgcc cctcctgccc | |
| 9451 ctcctgctcc tgcccctcct gcccctcctg ctcctgcccc tcctgctcct | |
| 9501 gcccctcctg ctcctgcccc tcctgctcct gcccctcctg cccctcctgc | |
| 9551 ccctcctcct gctcctgccc ctcctgctcc tgcccctcct gcccctcctg | |
| 9601 cccctcctgc tcctgcccct cctcctgctc ctgcccctcc tgcccctcct | |
| 9651 gcccctcctc ctgctcctgc ccctcctgcc cctcctcctg ctcctgcccc | |
| 9701 tcctcctgct cctgcccctc ctgcccctcc tgcccctcct cctgctcctg | |
| 9751 cccctcctgc ccctcctcct gctcctgccc ctcctcctgc tcctgcccct | |
| 9801 cctgcccctc ctgcccctcc tcctgctcct gcccctcctc ctgctcctgc | |
| 9851 ccctcctgcc cctcctgccc ctcctgcccc tcctcctgct cctgcccctc | |
| 9901 ctcctgctcc tgcccctcct gctcctgccc ctcccgctcc tgctcctgct | |
| 9951 cctgttccac cgtgggtccc tttgcagcca atgcaacttg gacgtttttg | |
| 10001 gggtctccgg acaccatctc tatgtcttgg ccctgatcct gagccgcccg | |
| 10051 gggctcctgg tcttccgcct cctcgtcctc gtcctcttcc ccgtcctcgt | |
| 10101 ccatggttat caccccctct tctttgaggt ccactgccgc cggagccttc | |
| 10151 tggtccagat gtgtctccct tctctcctag gccatttcca ggtcctgtac | |
| 10201 ctggcccctc gtcagacatg attcacacta aaagagatca atagacatct | |
| 10251 ttattagacg acgctcagtg aatacaggga gtgcagactc ctgccccctc | |
| 10301 caacagcccc cccaccctca tccccttcat ggtcgctgtc agacagatcc | |
| 10351 aggtctgaaa attccccatc ctccgaacca tcctcgtcct catcaccaat | |
| 10401 tactcgcagc ccggaaaact cccgctgaac atcctcaaga tttgcgtcct | |
| 10451 gagcctcaag ccaggcctca aattcctcgt cccccttttt gctggacggt | |
| 10501 agggatgggg attctcggga cccctcctct tcctcttcaa ggtcaccaga | |
| 10551 cagagatgct actggggcaa cggaagaaaa gctgggtgcg gcctgtgagg | |
| 10601 atcagcttat cgatgataag ctgtcaaaca tgagaattct tgaagacgaa | |
| 10651 agggcctcgt gatacgccta tttttatagg ttaatgtcat gataataatg | |
| 10701 gtttcttaga cgtcaggtgg cacttttcgg ggaaatgtgc gcggaacccc | |
| 10751 tatttgttta tttttctaaa tacattcaaa tatgtatccg ctcatgagac | |
| 10801 aataaccctg ataaatgctt caataatatt gaaaaaggaa gagtatgagt | |
| 10851 attcaacatt tccgtgtcgc ccttattccc ttttttgcgg cattttgcct | |
| 10901 tcctgttttt gctcacccag aaacgctggt gaaagtaaaa gatgctgaag | |
| 10951 atcagttggg tgcacgagtg ggttacatcg aactggatct caacagcggt | |
| 11001 aagatccttg agagttttcg ccccgaagaa cgttttccaa tgatgagcac | |
| 11051 ttttaaagtt ctgctatgtg gcgcggtatt atcccgtgtt gacgccgggc | |
| 11101 aagagcaact cggtcgccgc atacactatt ctcagaatga cttggttgag | |
| 11151 tactcaccag tcacagaaaa gcatcttacg gatggcatga cagtaagaga | |
| 11201 attatgcagt gctgccataa ccatgagtga taacactgcg gccaacttac | |
| 11251 ttctgacaac gatcggagga ccgaaggagc taaccgcttt tttgcacaac | |
| 11301 atgggggatc atgtaactcg ccttgatcgt tgggaaccgg agctgaatga | |
| 11351 agccatacca aacgacgagc gtgacaccac gatgcctgca gcaatggcaa | |
| 11401 caacgttgcg caaactatta actggcgaac tacttactct agcttcccgg | |
| 11451 caacaattaa tagactggat ggaggcggat aaagttgcag gaccacttct | |
| 11501 gcgctcggcc cttccggctg gctggtttat tgctgataaa tctggagccg | |
| 11551 gtgagcgtgg gtctcgcggt atcattgcag cactggggcc agatggtaag | |
| 11601 ccctcccgta tcgtagttat ctacacgacg gggagtcagg caactatgga | |
| 11651 tgaacgaaat agacagatcg ctgagatagg tgcctcactg attaagcatt | |
| 11701 ggtaactgtc agaccaagtt tactcatata tactttagat tgatttaaaa | |
| 11751 cttcattttt aatttaaaag gatctaggtg aagatccttt ttgataatct | |
| 11801 catgaccaaa atcccttaac gtgagttttc gttccactga gcgtcagacc | |
| 11851 ccgtagaaaa gatcaaagga tcttcttgag atcctttttt tctgcgcgta | |
| 11901 atctgctgct tgcaaacaaa aaaaccaccg ctaccagcgg tggtttgttt | |
| 11951 gccggatcaa gagctaccaa ctctttttcc gaaggtaact ggcttcagca | |
| 12001 gagcgcagat accaaatact gtccttctag tgtagccgta gttaggccac | |
| 12051 cacttcaaga actctgtagc accgcctaca tacctcgctc tgctaatcct | |
| 12101 gttaccagtg gctgctgcca gtggcgataa gtcgtgtctt accgggttgg | |
| 12151 actcaagacg atagttaccg gataaggcgc agcggtcggg ctgaacgggg | |
| 12201 ggttcgtgca cacagcccag cttggagcga acgacctaca ccgaactgag | |
| 12251 atacctacag cgtgagctat gagaaagcgc cacgcttccc gaagggagaa | |
| 12301 aggcggacag gtatccggta agcggcaggg tcggaacagg agagcgcacg | |
| 12351 agggagcttc cagggggaaa cgcctggtat ctttatagtc ctgtcgggtt | |
| 12401 tcgccacctc tgacttgagc gtcgattttt gtgatgctcg tcaggggggc | |
| 12451 ggagcctatg gaaaaacgcc agcaacgcgg cctttttacg gttcctggcc | |
| 12501 ttttgctggc cttgaagctg tccctgatgg tcgtcatcta cctgcctgga | |
| 12551 cagcatggcc tgcaacgcgg gcatcccgat gccgccggaa gcgagaagaa | |
| 12601 tcataatggg gaaggccatc cagcctcgcg tcgcgaacgc cagcaagacg | |
| 12651 tagcccagcg cgtcggcccc gagatgcgcc gcgtgcggct gctggagatg | |
| 12701 gcggacgcga tggatatgtt ctgccaaggg ttggtttgcg cattcacagt | |
| 12751 tctccgcaag aattgattgg ctccaattct tggagtggtg aatccgttag | |
| 12801 cgaggtgccg ccctgcttca tccccgtggc ccgttgctcg cgtttgctgg | |
| 12851 cggtgtcccc ggaagaaata tatttgcatg tctttagttc tatgatgaca | |
| 12901 caaaccccgc ccagcgtctt gtcattggcg aattcgaaca cgcagatgca | |
| 12951 gtcggggcgg cgcggtccga ggtccacttc gcatattaag gtgacgcgtg | |
| 13001 tggcctcgaa caccgagcga ccctgcagcg acccgcttaa cagcgtcaac | |
| 13051 agcgtgccgc agatcccggg gggcaatgag atatgaaaaa gcctgaactc | |
| 13101 accgcgacgt ctgtcgagaa gtttctgatc gaaaagttcg acagcgtctc | |
| 13151 cgacctgatg cagctctcgg agggcgaaga atctcgtgct ttcagcttcg | |
| 13201 atgtaggagg gcgtggatat gtcctgcggg taaatagctg cgccgatggt | |
| 13251 ttctacaaag atcgttatgt ttatcggcac tttgcatcgg ccgcgctccc | |
| 13301 gattccggaa gtgcttgaca ttggggaatt cagcgagagc ctgacctatt | |
| 13351 gcatctcccg ccgtgcacag ggtgtcacgt tgcaagacct gcctgaaacc | |
| 13401 gaactgcccg ctgttctgca gccggtcgcg gaggccatgg atgcgatcgc | |
| 13451 tgcggccgat cttagccaga cgagcgggtt cggcccattc ggaccgcaag | |
| 13501 gaatcggtca atacactaca tggcgtgatt tcatatgcgc gattgctgat | |
| 13551 ccccatgtgt atcactggca aactgtgatg gacgacaccg tcagtgcgtc | |
| 13601 cgtcgcgcag gctctcgatg agctgatgct ttgggccgag gactgccccg | |
| 13651 aagtccggca cctcgtgcac gcggatttcg gctccaacaa tgtcctgacg | |
| 13701 gacaatggcc gcataacagc ggtcattgac tggagcgagg cgatgttcgg | |
| 13751 ggattcccaa tacgaggtcg ccaacatctt cttctggagg ccgtggttgg | |
| 13801 cttgtatgga gcagcagacg cgctacttcg agcggaggca tccggagctt | |
| 13851 gcaggatcgc cgcggctccg ggcgtatatg ctccgcattg gtcttgacca | |
| 13901 actctatcag agcttggttg acggcaattt cgatgatgca gcttgggcgc | |
| 13951 agggtcgatg cgacgcaatc gtccgatccg gagccgggac tgtcgggcgt | |
| 14001 acacaaatcg cccgcagaag cgcggccgtc tggaccgatg gctgtgtaga | |
| 14051 agtactcgcc gatagtggaa accgacgccc cagcactcgt ccggatcggg | |
| 14101 agatggggga ggctaactga aacacggaag gagacaatac cggaaggaac | |
| 14151 ccgcgctatg acggcaataa aaagacagaa taaaacgcac gggtgttggg | |
| 14201 tcgtttgttc ataaacgcgg ggttcggtcc cagggctggc actctgtcga | |
| 14251 taccccaccg agaccccatt ggggccaata cgcccgcgtt tcttcctttt | |
| 14301 ccccacccca ccccccaagt tcgggtgaag gcccagggct cgcagccaac | |
| 14351 gtcggggcgg caggccctgc catagccact ggccccgtgg gttagggacg | |
| 14401 gggtccccca tggggaatgg tttatggttc gtgggggtta ttattttggg | |
| 14451 cgttgcgtgg ggtcaggtcc acgactggac tgagcagaca gacccatggt | |
| 14501 ttttggatgg cctgggcatg gaccgcatgt actggcgcga cacgaacacc | |
| 14551 gggcgtctgt ggctgccaaa cacccccgac ccccaaaaac caccgcgcgg | |
| 14601 atttctggcg tgccaagcta | |
| Sequence of expression cassette GD532-GLuc (SEQ ID NO: 2) | |
| tgggtggggc actttaggac tgtggttcat ttgaattggt gtaaacaata caccggttct | 60 |
| actgtcctac agcctccatt cagatgactg aagtcatggg actttcagca tagctagctg | 120 |
| atgacagtgc atactatttt gtcccaaaat ccagttcaag catggacata ccaataagag | 180 |
| cctaagctct ttaaaggcaa aggaccagga attgtacagt tcttggtata gaagaagaca | 240 |
| ggcaaaagtg tttttgaact aacgttaaat gtgcaatatg ttagaattca tgcaatgcac | 300 |
| aggactgcag gattctgata tcttatttaa ctctcaaatt ctattcaact caataaacct | 360 |
| tgactgtgct tctactaaat gcaggtattg tactaggagc tgaggacacc aaactgatga | 420 |
| agtccttgct gtcaagaaac tcacatgatt ccctaattct ttgtcagctt gctgtgatca | 480 |
| cattttcttc ccaagaacct ctaagaaatg cctagtggat agaaccttgg agttccacgg | 540 |
| aacatattaa caatcgccaa atgatgactc aggctagatt gtgtaattca ggttttgtct | 600 |
| gcaaaactga aaatgcttcg gtaacctacc taaatttcaa tgttgaggaa ttctttaaga | 660 |
| aagacatcaa atgttaagat ttaaggcata gatatgagat acatagtcat gcttaggtga | 720 |
| attatgcact gaccatgacc atttctttac tcaaatgttg tccatggctg acaacacagt | 780 |
| gaaaaaatga gtgcaaaatg acaactcaaa taaatgaacc agaaaaccta tcacttttct | 840 |
| tttccaccaa attaagatca agagagctgg agaatatttt gtctagagtg ataaaaacat | 900 |
| aagggtgcaa aacttccagg tacctttgca gaaattactt ctgtgacctt tggctgtaca | 960 |
| gcaaccttaa taatgcaagc actgttttga atgcaagcat gtgggagcca ttttcaccac | 1020 |
| ttttgatgac ttcagtaggt ttaagaaatg tttttgcttt tattgcataa accataaaac | 1080 |
| aaaggaaggg acttttgaac tactcagtga gagtctatat attaaagttt gtttttcaaa | 1140 |
| aatgtgtaac taccatttgc agttttaaag gtctgctttc cacctacaag ttgccattat | 1200 |
| ctcaaaggtg aaattttagc atatgactaa aaacttccta tagttacagc ttcatgattc | 1260 |
| agcatctaac atcaataatt cacagtgaga tcataggagg ctctctgtgg aaggtaacga | 1320 |
| catacatacg ttaggaaagg aagcttaggg catatcgaga gcattttgaa tttagacttg | 1380 |
| tgggctgtgt gggtgtcaga tggttgtctc tcagctggtg ggcgtccaga aggatccttg | 1440 |
| tttgggcaag gctctttgag aaaggagaat ctgggttgcc agggattccc acatgtggtc | 1500 |
| accagctccc cacgcagacc agctcacgat ttcccagtta caccgggcag gtgggaaacc | 1560 |
| gttctgcttt ctgtggaaaa gattctaact tggttccctg ccatccctga atacaaacgg | 1620 |
| gttggttttt cttttttgag cttccaaccc ttgcagcttt ccaaaaataa atcaaaccag | 1680 |
| ccatcagggc accgaaataa tactactgct aataagcagc ttcgcctaga cttagataaa | 1740 |
| caacacttct gaggtaaact ttgccccgga ggtctggaga cactttttta atgtaacctg | 1800 |
| cttactaata attactagac ttcagtgcat taaccctgga aatagatttt aatagccacc | 1860 |
| ccttaaaaca aaagacatga aaagataata agaaaaaagt gccgcaacta ttatagaaaa | 1920 |
| acacttggca gcctgcttca gcccaagctg aggccacctc tagcctctgc taaagccccc | 1980 |
| cactcccaat ggtccccgcc aaccggataa gagtgcgcgc gggacccgcc ttcccctctc | 2040 |
| ggcaccgccc ccgcccccgc cccctcggct cgcctcccgc gtggctcctc ccttttccgc | 2100 |
| tcctctcaac ctgactccag gagctggggt caaattgctg gagcaggctg atttgcatag | 2160 |
| cccaatggcc aagctgcatg caaatgaggc ggaaggtggt tggctgaggg ttggcaggat | 2220 |
| aaccccggag agcggggccc tttgtcctcc agtggctggt aggcagtggc tgggaggcag | 2280 |
| cggcccaatt agtgtcgtgc ggcccgtggc gaggcgaggt ccggggagcg agcgagcaag | 2340 |
| caaggcggga ggggtggccg gagctgcggc ggctggcaca ggaggaggag cccgggcggg | 2400 |
| cgaggggcgg ccggagagcg ccagggcctg agctgccgga gcggcgcctg tgagtgagtg | 2460 |
| cagaaagcag gcgcccgcgc gctagccgtg gcaggagcag cccgcacgcc gcgctctctc | 2520 |
| cctgggcgac ctgcagtttg caatatggga gtcaaagttc tgtttgccct gatctgcatc | 2580 |
| gctgtggccg aggccaagcc caccgagaac aacgaagact tcaacatcgt ggccgtggcc | 2640 |
| agcaacttcg cgaccacgga tctcgatgct gaccgcggga agttgcccgg caagaagctg | 2700 |
| ccgctggagg tgctcaaaga gatggaagcc aatgcccgga aagctggctg caccaggggc | 2760 |
| tgtctgatct gcctgtccca catcaagtgc acgcccaaga tgaagaagtt catcccagga | 2820 |
| cgctgccaca cctacgaagg cgacaaagag tccgcacagg gcggcatagg cgaggcgatc | 2880 |
| gtcgacattc ctgagattcc tgggttcaag gacttggagc ccatggagca gttcatcgca | 2940 |
| caggtcgatc tgtgtgtgga ctgcacaact ggctgcctca aagggcttgc caacgtgcag | 3000 |
| tgttctgacc tgctcaagaa gtggctgccg caacgctgtg cgacctttgc cagcaagatc | 3060 |
| cagggccagg tggacaagat caagggggcc ggtggtgact aatgcggccg cgactctaga | 3120 |
| tcataatcag ccataccaca tttgtagagg ttttacttgc tttaaaaaac ctcccacacc | 3180 |
| tccccctgaa cctgaaacat aaaatgaatg caattgttgt tgttatcgcg accccgataa | 3240 |
| cgtggtgttg tgcctgctgg cggcggacga ggacgacgac agagatgtgg ctctgcagat | 3300 |
| ccacttcacc ctgatccagg cgttttgctg cgagaacgac atcaacatcc tgcgcgtcag | 3360 |
| caacccgggc cggctggcgg agctcctgct cttggagacc gacgctggcc ccgcggcgag | 3420 |
| cgagggcgcc gagcagcccc cggacctgca ctgcgtgctg gtgacggtaa gggactgggg | 3480 |
| gactgcagcc tgcagggtag agccccggaa ggacgggagt caggactggg ttgcctgatt | 3540 |
| gtggatctgt ggtaggtgag ggtcaggagg gtggctgcct ttgcccgact agagtgtggc | 3600 |
| tggactttca gccgagatgt gctagtttca tcatcaggat tttctgtggt acagaacatg | 3660 |
| tctaagcatg ctggggactg ccagcagcgg aagagatccc tgtgagtcag cagtcagccc | 3720 |
| agctactccc tacctacatc tgcactgcct cccgtgacta attcctttag cagggcagat | 3780 |
| tagataaagc caaatgaatt cctggctcac ccctcattaa ggagtcagct tcattctctg | 3840 |
| ccagtcagag ctaaaaatag aaattgtgta ggagacaaac cttgttaatt ccctagaaat | 3900 |
| acattaagag gatagagtgg aatttttttt ctctgcaatc ttgcattttt ttaatggctc | 3960 |
| tttttttttt tcctgataaa aacctttggt aggtagggaa gttatgtttt caggggtaaa | 4020 |
| tgtgctactt ttgtcttcta aattttgctc ttttttgact ggtctagtca agtgacagcc | 4080 |
| cgattatttt gctactcctt aaaagtacta ttctgtctct tggagtatgg ttgatggcaa | 4140 |
| ttccagttaa ctgctgtgca gctctcatct cattgtgcac acagcatgga aatctttctc | 4200 |
| aaaactgttt cactcaggtc agggtaacaa gtttggtaga gcaaaccggt gaatgatact | 4260 |
| ctcatgcaaa actgaacaga tatgcaaaca tatgtatgtg gttcagcttg ggttgcatgg | 4320 |
| gttcagactt tgcaatgtgt agtttaatag gtaattaccc ttaacgcttt tgcagggaac | 4380 |
| ccaactacct tgaagaaact ttaatttttt tgtgcttcta atttgtctcc atgtcacata | 4440 |
| gccaaaatat agaatgttca agtgttttct cctcaaaagt ataattacta gaatatactg | 4500 |
| gtttttaaaa taagtttatt tttataaatt tgtttccaga atccacattc atctcaatgg | 4560 |
| aaggatcctg ccttaagtca acttatttgt ttttgccggg aaagtcgcta catggatcaa | 4620 |
| tgggttccag tgattaatct ccctgaacgg tgatggcatc tgaatgaaaa taactgaacc | 4680 |
| aaattgcact gaagtttttg aaataccttt gtagttactc aagcagttac tccctacact | 4740 |
| gatgcaagga ttacagaaac tgatgccaag gggctgagtg agttcaacta catgttctgg | 4800 |
| gggcccggag atagatgact ttgcagatgg aaagaggtga aaatgaagaa ggaagctgtg | 4860 |
| ttgaaacaga aaaataagtc aaaaggaaca aaaattacaa agaaccatgc aggaaggaaa | 4920 |
| actatgtatt aatttagaat ggttgagtta cattaaaata aaccaaatat gttaaagttt | 4980 |
| aagtgtgcag ccatagtttg ggtatttttg gtttatatgc cctcaagtaa aagaaaagcc | 5040 |
| gaaagggtta atcatatttg aaaaccatat tttattgtat tttgatgaga tattaaattc | 5100 |
| tcaaagtttt attataaatt ctactaagtt attttatgac atgaaaagtt atttatgcta | 5160 |
| taaatttttt gaaacacaat acctacaata aactggtatg aataattgca tcatt | 5215 |

1. An expression cassette comprising a DNA sequence encoding Gaussia luciferase (GLuc) reporter protein and derivatives thereof, which DNA sequence is operatively linked to a human GADD45α gene promoter and a human GADD45α gene regulatory element arranged to activate expression of the DNA sequence encoding Gaussia luciferase (GLuc) reporter protein in response to genome damage.

2. The expression cassette of claim 1, wherein the regulatory element comprises Exon 1, Exon 2, Exon 3, and/or Exon 4 of the GADD45α gene, or at least a region thereof, or any combination thereof.

3. The expression cassette of claim 2, wherein the regulatory element comprises at least a region of Exon 1 of the GADD45α gene, at least a region of Exon 3 of the GADD45α gene, and at least a region of Exon 4 of the GADD45α gene.

4. The expression cassette of claim 1, wherein the regulatory element comprises Intron 1, Intron 2, and/or Intron 3 of the GADD45α gene, or at least a region thereof, or any combination thereof.

5. The expression cassette of claim 4, wherein the regulatory element comprises at least a region of Intron 3 of the GADD45α gene.

6. The expression cassette of claim 5, wherein the regulatory element comprises a putative p53 binding motif.

7. The expression cassette of claim 5, wherein the regulatory element comprises a putative AP-1 motif.

8. The expression cassette of claim 1, wherein the genome damage is DNA damage.

9. The expression cassette of claim 1, wherein the DNA sequence encoding Gaussia luciferase (GLuc) is shown at positions 2641-3198 of SEQ ID NO:1

10. The expression cassette of claim 1, which is GD532-GLuc, designated as SEQ ID NO:2.

11. A recombinant vector comprising the expression cassette of claim 1.

12. The recombinant vector of claim 11, which is pEP-GD532-GLuc, designated as SEQ ID NO:1.

13. A cell containing the expression cassette of claim 1 or a recombinant vector comprising the expression cassette.

14. The cell of claim 13, wherein the cell is a human cell.

15. The cell of claim 14, wherein the cell is a human cell having a fully functional p53.

16. The cell of claim 15, wherein the cell is a TK6 human cell line.

17. A method of detecting for the presence of an agent that causes or potentiates genome damage comprising subjecting a cell that contains an expression cassette comprising a DNA sequence encoding Gaussia luciferase (GLuc) reporter protein and derivatives thereof, which DNA sequence is operatively linked to a human GADD45α gene promoter and a human GADD45α gene regulatory element arranged to activate expression of the DNA sequence encoding Gaussia luciferase (GLuc) reporter protein in response to genome damage, or a recombinant vector containing the expression cassette, to an agent; and monitoring the expression of the DNA sequence encoding the GLuc reporter protein from the cell.

18. The method of claim 17, wherein the agent is further screened to assess whether it is safe to expose a living organism to the agent.

19. The method of claim 17, wherein the agent is a candidate medicament, food additive or cosmetic.

20. The method of claim 17, comprising preparing a population of the cells; incubating the cells with the agent for a pre-determined time; and monitoring the expression of the DNA sequence encoding the GLuc reporter protein directly from a sample of the cells.

21. The method of claim 20, wherein the method is performed in the presence of S9 liver extracts.

22. The method of claim 21, wherein the density of the cells in the population is determined using a cell stain.

23. The method of claim 22, wherein the cell stain is a cyanine dye.

24. The method of claim 23, wherein the cyanine dye is thiazole orange.

25. The method of claim 17, wherein the expression of the GLuc reporter protein is monitored after between 46 to 50 hours from exposure to the test compound.


SEQUENCE LISTING (2 sequences)

SEQ ID NO: 1 - DNA, 14620 bp, Artificial
  Sequence of pEP-GD532 GLuc
gtcgaccaattctcatgtttgacagcttatcatcgcagatccgggcaacgttgttgccattgctgcaggcgcagaactggtaggtatggaagatcttgggtggggcactttaggactgtggttcatttgaattggtgtaaacaatacaccggttctactgtcctacagcctccattcagatgactgaagtcatgggactttcagcatagctagctgatgacagtgcatactattttgtcccaaaatccagttcaagcatggacataccaataagagcctaagctctttaaaggcaaaggaccaggaattgtacagttcttggtatagaagaagacaggcaaaagtgtttttgaactaacgttaaatgtgcaatatgttagaattcatgcaatgcacaggactgcaggattctgatatcttatttaactctcaaattctattcaactcaataaaccttgactgtgcttctactaaatgcaggtattgtactaggagctgaggacaccaaactgatgaagtccttgctgtcaagaaactcacatgattccctaattctttgtcagcttgctgtgatcacattttcttcccaagaacctctaagaaatgcctagtggatagaaccttggagttccacggaacatattaacaatcgccaaatgatgactcaggctagattgtgtaattcaggttttgtctgcaaaactgaaaatgcttcggtaacctacctaaatttcaatgttgaggaattctttaagaaagacatcaaatgttaagatttaaggcatagatatgagatacatagtcatgcttaggtgaattatgcactgaccatgaccatttctttactcaaatgttgtccatggctgacaacacagtgaaaaaatgagtgcaaaatgacaactcaaataaatgaaccagaaaacctatcacttttcttttccaccaaattaagatcaagagagctggagaatattttgtctagagtgataaaaacataagggtgcaaaacttccaggtacctttgcagaaattacttctgtgacctttggctgtacagcaaccttaataatgcaagcactgttttgaatgcaagcatgtgggagccattttcaccacttttgatgacttcagtaggtttaagaaatgtttttgcttttattgcataaaccataaaacaaaggaagggacttttgaactactcagtgagagtctatatattaaagtttgtttttcaaaaatgtgtaactaccatttgcagttttaaaggtctgctttccacctacaagttgccattatctcaaaggtgaaattttagcatatgactaaaaacttcctatagttacagcttcatgattcagcatctaacatcaataattcacagtgagatcataggaggctctctgtggaaggtaacgacatacatacgttaggaaaggaagcttagggcatatcgagagcattttgaatttagacttgtgggctgtgtgggtgtcagatggttgtctctcagctggtgggcgtccagaaggatccttgtttgggcaaggctctttgagaaaggagaatctgggttgccagggattcccacatgtggtcaccagctccccacgcagaccagctcacgatttcccagttacaccgggcaggtgggaaaccgttctgctttctgtggaaaagattctaacttggttccctgccatccctgaatacaaacgggttggtttttcttttttgagcttccaacccttgcagctttccaaaaataaatcaaaccagccatcagggcaccgaaataatactactgctaataagcagcttcgcctagacttagataaacaacacttctgaggtaaactttgccccggaggtctggagacacttttttaatgtaacctgcttactaataattactagacttcagtgcattaaccctggaaatagattttaatagccaccccttaaaacaaaagacatgaaaagataataagaaaaaagtgccgcaactattatagaaaaacacttggcagcctgcttcagcccaagctgaggccacctctagcctctgctaaagccccccactcccaatggtccccgccaaccggataagagtgcgcgcgggacccgccttcccctctcggcaccgcccccgcccccgccccctcggctcgcctcccgcgtggctcctcccttttccgctcctctcaacctgactccaggagctggggtcaaattgctggagcaggctgatttgcatagcccaatggccaagctgcatgcaaatgaggcggaaggtggttggctgagggttggcaggataaccccggagagcggggccctttgtcctccagtggctggtaggcagtggctgggaggcagcggcccaattagtgtcgtgcggcccgtggcgaggcgaggtccggggagcgagcgagcaagcaaggcgggaggggtggccggagctgcggcggctggcacaggaggaggagcccgggcgggcgaggggcggccggagagcgccagggcctgagctgccggagcggcgcctgtgagtgagtgcagaaagcaggcgcccgcgcgctagccgtggcaggagcagcccgcacgccgcgctctctccctgggcgacctgcagtttgcaatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaatgcggccgcgactctagatcataatcagccataccacatttgtagaggttttacttgctttaaaaaacctcccacacctccccctgaacctgaaacataaaatgaatgcaattgttgttgttatcgcgaccccgataacgtggtgttgtgcctgctggcggcggacgaggacgacgacagagatgtggctctgcagatccacttcaccctgatccaggcgttttgctgcgagaacgacatcaacatcctgcgcgtcagcaacccgggccggctggcggagctcctgctcttggagaccgacgctggccccgcggcgagcgagggcgccgagcagcccccggacctgcactgcgtgctggtgacggtaagggactgggggactgcagcctgcagggtagagccccggaaggacgggagtcaggactgggttgcctgattgtggatctgtggtaggtgagggtcaggagggtggctgcctttgcccgactagagtgtggctggactttcagccgagatgtgctagtttcatcatcaggattttctgtggtacagaacatgtctaagcatgctggggactgccagcagcggaagagatccctgtgagtcagcagtcagcccagctactccctacctacatctgcactgcctcccgtgactaattcctttagcagggcagattagataaagccaaatgaattcctggctcacccctcattaaggagtcagcttcattctctgccagtcagagctaaaaatagaaattgtgtaggagacaaaccttgttaattccctagaaatacattaagaggatagagtggaattttttttctctgcaatcttgcatttttttaatggctctttttttttttcctgataaaaacctttggtaggtagggaagttatgttttcaggggtaaatgtgctacttttgtcttctaaattttgctcttttttgactggtctagtcaagtgacagcccgattattttgctactccttaaaagtactattctgtctcttggagtatggttgatggcaattccagttaactgctgtgcagctctcatctcattgtgcacacagcatggaaatctttctcaaaactgtttcactcaggtcagggtaacaagtttggtagagcaaaccggtgaatgatactctcatgcaaaactgaacagatatgcaaacatatgtatgtggttcagcttgggttgcatgggttcagactttgcaatgtgtagtttaataggtaattacccttaacgcttttgcagggaacccaactaccttgaagaaactttaatttttttgtgcttctaatttgtctccatgtcacatagccaaaatatagaatgttcaagtgttttctcctcaaaagtataattactagaatatactggtttttaaaataagtttatttttataaatttgtttccagaatccacattcatctcaatggaaggatcctgccttaagtcaacttatttgtttttgccgggaaagtcgctacatggatcaatgggttccagtgattaatctccctgaacggtgatggcatctgaatgaaaataactgaaccaaattgcactgaagtttttgaaatacctttgtagttactcaagcagttactccctacactgatgcaaggattacagaaactgatgccaaggggctgagtgagttcaactacatgttctgggggcccggagatagatgactttgcagatggaaagaggtgaaaatgaagaaggaagctgtgttgaaacagaaaaataagtcaaaaggaacaaaaattacaaagaaccatgcaggaaggaaaactatgtattaatttagaatggttgagttacattaaaataaaccaaatatgttaaagtttaagtgtgcagccatagtttgggtatttttggtttatatgccctcaagtaaaagaaaagccgaaagggttaatcatatttgaaaaccatattttattgtattttgatgagatattaaattctcaaagttttattataaattctactaagttattttatgacatgaaaagttatttatgctataaattttttgaaacacaatacctacaataaactggtatgaataattgcatcatttcttattgtgtgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagcta

SEQ ID NO: 2 - DNA, 5215 bp, Artificial
  Sequence of expression cassette GD532-GLuc
tgggtggggcactttaggactgtggttcatttgaattggtgtaaacaatacaccggttctactgtcctacagcctccattcagatgactgaagtcatgggactttcagcatagctagctgatgacagtgcatactattttgtcccaaaatccagttcaagcatggacataccaataagagcctaagctctttaaaggcaaaggaccaggaattgtacagttcttggtatagaagaagacaggcaaaagtgtttttgaactaacgttaaatgtgcaatatgttagaattcatgcaatgcacaggactgcaggattctgatatcttatttaactctcaaattctattcaactcaataaaccttgactgtgcttctactaaatgcaggtattgtactaggagctgaggacaccaaactgatgaagtccttgctgtcaagaaactcacatgattccctaattctttgtcagcttgctgtgatcacattttcttcccaagaacctctaagaaatgcctagtggatagaaccttggagttccacggaacatattaacaatcgccaaatgatgactcaggctagattgtgtaattcaggttttgtctgcaaaactgaaaatgcttcggtaacctacctaaatttcaatgttgaggaattctttaagaaagacatcaaatgttaagatttaaggcatagatatgagatacatagtcatgcttaggtgaattatgcactgaccatgaccatttctttactcaaatgttgtccatggctgacaacacagtgaaaaaatgagtgcaaaatgacaactcaaataaatgaaccagaaaacctatcacttttcttttccaccaaattaagatcaagagagctggagaatattttgtctagagtgataaaaacataagggtgcaaaacttccaggtacctttgcagaaattacttctgtgacctttggctgtacagcaaccttaataatgcaagcactgttttgaatgcaagcatgtgggagccattttcaccacttttgatgacttcagtaggtttaagaaatgtttttgcttttattgcataaaccataaaacaaaggaagggacttttgaactactcagtgagagtctatatattaaagtttgtttttcaaaaatgtgtaactaccatttgcagttttaaaggtctgctttccacctacaagttgccattatctcaaaggtgaaattttagcatatgactaaaaacttcctatagttacagcttcatgattcagcatctaacatcaataattcacagtgagatcataggaggctctctgtggaaggtaacgacatacatacgttaggaaaggaagcttagggcatatcgagagcattttgaatttagacttgtgggctgtgtgggtgtcagatggttgtctctcagctggtgggcgtccagaaggatccttgtttgggcaaggctctttgagaaaggagaatctgggttgccagggattcccacatgtggtcaccagctccccacgcagaccagctcacgatttcccagttacaccgggcaggtgggaaaccgttctgctttctgtggaaaagattctaacttggttccctgccatccctgaatacaaacgggttggtttttcttttttgagcttccaacccttgcagctttccaaaaataaatcaaaccagccatcagggcaccgaaataatactactgctaataagcagcttcgcctagacttagataaacaacacttctgaggtaaactttgccccggaggtctggagacacttttttaatgtaacctgcttactaataattactagacttcagtgcattaaccctggaaatagattttaatagccaccccttaaaacaaaagacatgaaaagataataagaaaaaagtgccgcaactattatagaaaaacacttggcagcctgcttcagcccaagctgaggccacctctagcctctgctaaagccccccactcccaatggtccccgccaaccggataagagtgcgcgcgggacccgccttcccctctcggcaccgcccccgcccccgccccctcggctcgcctcccgcgtggctcctcccttttccgctcctctcaacctgactccaggagctggggtcaaattgctggagcaggctgatttgcatagcccaatggccaagctgcatgcaaatgaggcggaaggtggttggctgagggttggcaggataaccccggagagcggggccctttgtcctccagtggctggtaggcagtggctgggaggcagcggcccaattagtgtcgtgcggcccgtggcgaggcgaggtccggggagcgagcgagcaagcaaggcgggaggggtggccggagctgcggcggctggcacaggaggaggagcccgggcgggcgaggggcggccggagagcgccagggcctgagctgccggagcggcgcctgtgagtgagtgcagaaagcaggcgcccgcgcgctagccgtggcaggagcagcccgcacgccgcgctctctccctgggcgacctgcagtttgcaatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaatgcggccgcgactctagatcataatcagccataccacatttgtagaggttttacttgctttaaaaaacctcccacacctccccctgaacctgaaacataaaatgaatgcaattgttgttgttatcgcgaccccgataacgtggtgttgtgcctgctggcggcggacgaggacgacgacagagatgtggctctgcagatccacttcaccctgatccaggcgttttgctgcgagaacgacatcaacatcctgcgcgtcagcaacccgggccggctggcggagctcctgctcttggagaccgacgctggccccgcggcgagcgagggcgccgagcagcccccggacctgcactgcgtgctggtgacggtaagggactgggggactgcagcctgcagggtagagccccggaaggacgggagtcaggactgggttgcctgattgtggatctgtggtaggtgagggtcaggagggtggctgcctttgcccgactagagtgtggctggactttcagccgagatgtgctagtttcatcatcaggattttctgtggtacagaacatgtctaagcatgctggggactgccagcagcggaagagatccctgtgagtcagcagtcagcccagctactccctacctacatctgcactgcctcccgtgactaattcctttagcagggcagattagataaagccaaatgaattcctggctcacccctcattaaggagtcagcttcattctctgccagtcagagctaaaaatagaaattgtgtaggagacaaaccttgttaattccctagaaatacattaagaggatagagtggaattttttttctctgcaatcttgcatttttttaatggctctttttttttttcctgataaaaacctttggtaggtagggaagttatgttttcaggggtaaatgtgctacttttgtcttctaaattttgctcttttttgactggtctagtcaagtgacagcccgattattttgctactccttaaaagtactattctgtctcttggagtatggttgatggcaattccagttaactgctgtgcagctctcatctcattgtgcacacagcatggaaatctttctcaaaactgtttcactcaggtcagggtaacaagtttggtagagcaaaccggtgaatgatactctcatgcaaaactgaacagatatgcaaacatatgtatgtggttcagcttgggttgcatgggttcagactttgcaatgtgtagtttaataggtaattacccttaacgcttttgcagggaacccaactaccttgaagaaactttaatttttttgtgcttctaatttgtctccatgtcacatagccaaaatatagaatgttcaagtgttttctcctcaaaagtataattactagaatatactggtttttaaaataagtttatttttataaatttgtttccagaatccacattcatctcaatggaaggatcctgccttaagtcaacttatttgtttttgccgggaaagtcgctacatggatcaatgggttccagtgattaatctccctgaacggtgatggcatctgaatgaaaataactgaaccaaattgcactgaagtttttgaaatacctttgtagttactcaagcagttactccctacactgatgcaaggattacagaaactgatgccaaggggctgagtgagttcaactacatgttctgggggcccggagatagatgactttgcagatggaaagaggtgaaaatgaagaaggaagctgtgttgaaacagaaaaataagtcaaaaggaacaaaaattacaaagaaccatgcaggaaggaaaactatgtattaatttagaatggttgagttacattaaaataaaccaaatatgttaaagtttaagtgtgcagccatagtttgggtatttttggtttatatgccctcaagtaaaagaaaagccgaaagggttaatcatatttgaaaaccatattttattgtattttgatgagatattaaattctcaaagttttattataaattctactaagttattttatgacatgaaaagttatttatgctataaattttttgaaacacaatacctacaataaactggtatgaataattgcatcatt